(19) United States
(12) Patent Application Publication
(10) Pub. No.: US 2005/0189873 A1
(43) Pub. Date: Sep. 1, 2005

(54) Lighting elements, devices and methods

(76) Inventors: Stephen M. Kelly, Beverley (GB); Mary O'Neill, Hull (GB); Gene C. Koch, Bayville, NJ (US)

Correspondence Address: Ronald D. Trice, 2101 Crystal Plaza Arcade 138, Arlington, VA, 22202-4600, US

(21) Appl. No.: 10/948,748

(22) Filed: Sep. 24, 2004

Related U.S. Application Data
(60) Provisional application No. 60/563,343, filed on Apr. 16, 2004.
(60) Provisional application No. 60/505,446, filed on Sep. 25, 2003.

(51) Int. Cl. H01J 1/62; C08F 271/02; C07D 211/80

(52) U.S. Cl. 313/504; 544/294; 546/285; 525/279

(57) ABSTRACT

The invention relates to liquid crystalline emitter and charge-transport materials for use in organic light emitting devices. These materials may be used as uncrosslinked liquid crystalline glasses or crosslinked as insoluble polymer matrices. The polymer may be formed by photopolymerization. The polymerization may be done without a photoinitiator. The polymer may have a room temperature nematic phase that may be stabilized the nematic phase relative to smectic phases. The polymer may be easily photocrosslinked with a high final degree of polymerization. The layers of crosslinked layers organic semiconductor may be incorporated into electronic devices. The materials have a high luminous output. Exemplary light emitting polymer may be formed by the polymerization of a reactive mesogen having the formula: B₁—S₁-T₁-(F-T₂)ₚ-F-T₃-S₂—B₂, wherein B₁ and B₂ are polymerizable end groups, F is a fluorene functional unit, S₁ and S₂ are spacer units, and T₁, T₂, and T₃ are thienothiophenes units.

### RELATED APPLICATIONS

This application claims priority from, and incorporates by reference, U.S. Provisional application Ser. No. 60/563,343, filed Apr. 16, 2004, and U.S. Provisional application Ser. No. 60/505,446, filed Sep. 25, 2003.

### FIELD OF THE INVENTION

The present invention relates generally to materials for use in organic light emitting devices (OLEDs); and more particularly, to liquid crystalline emitter and charge-transport materials for use in OLEDs. The present invention also relates generally to thienothiophene containing organic semiconductor compositions, fabrication methods and devices, and more particularly, to polymer networks formed from mixtures of reactive mesogens, methods of fabricating polymer networks formed from mixtures of reactive mesogens and devices including polymer networks formed from mixtures of reactive mesogens.

### BACKGROUND

Organic light emitting devices that include liquid crystalline semiconductors are able to produce polarized light. These semiconductors have a number of properties that affect the performance and useful life of the organic light emitting devices. For example, crosslinkable liquid crystalline semiconductors containing fused polycyclic thienothiophene have some good properties but also may have high melting points which complicate device manufacture, poor alignment, and lower crosslink densities. When this semiconductor has crosslinking moieties that include, for example, acrylate groups, there is substantial film shrinkage on curing and substantial photodegradation that compromises performance as both a charge carrier transport medium and as an emissive material. When this semiconductor has crosslinking moieties that include oxetanes groups, a cationic (Lewis acid) initiator is used to initiate crosslinking. The initiator remains in the crosslinked polymer may have an adverse impact on the operating life of the devices fabricated from the semiconductor. According, there is a strong need in the art for room-temperature semiconductors that may be easily crosslinked with a high final degree of polymerization yielding layers of uniformly aligned organic semiconductor polymer having operating lifetimes uncompromised by the polymerization process.

### SUMMARY OF THE INVENTION

An exemplary compound according to the present invention includes the following structural units: 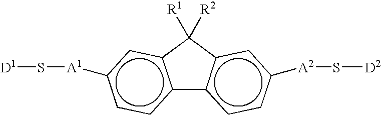
wherein either A¹ or A² or both are of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula 1.1:

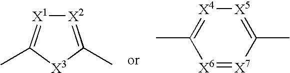

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 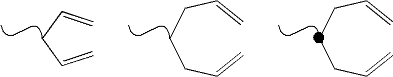
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

An exemplary for forming a light emitting polymer according to the present invention includes photopolymerization of a reactive mesogen having the formula: 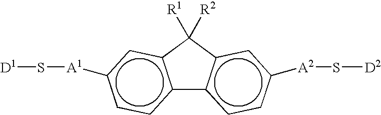
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula 1.1:

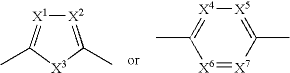

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 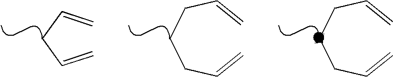
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO, —OCO—, —OCO—O—, —S—CO—, —CO—S—, —CH═CH—, —C≡C— such that O and S atoms are not directly linked to other O or S atoms.

Another exemplary process for forming a light emitting polymer according to the present invention including photopolymerization of a reactive mesogen mixture composed of two more components having the formula: 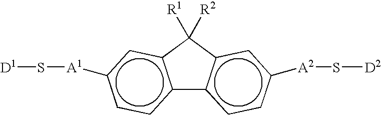
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula 1.1:

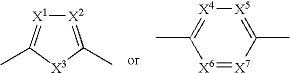

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 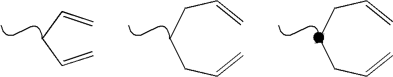
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The mixture may be a thermodynamically stable liquid crystal phase at room temperature.

Another exemplary process for forming a polymeric charge carrier transport layer according to the present invention includes photopolymerization of a reactive mesogen having the formula: 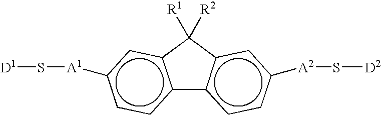
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula 1.1:

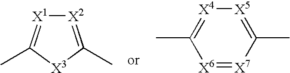

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 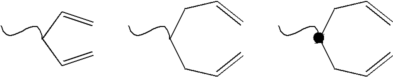
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —CO, —S—CO—, —CO—S—, —CH═CH—, —C≡C— such that O and S atoms are not directly linked to other O or S atoms.

Another process for forming a polymeric charge carrier transport layer according to the present invention includes photopolymerization of a reactive mesogen mixture composed of two more components having the formula: 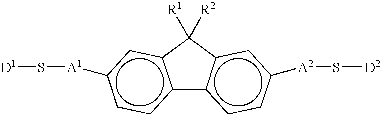
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula 1.1:

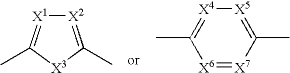

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 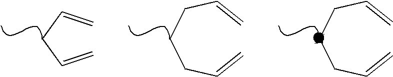
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The mixture may be a thermodynamically stable liquid crystal phase at room temperature. The light emitting polymer may be in the form of a liquid crystal and may be aligned to emit polarized light.

Another exemplary process for applying a light emitting polymer to a surface according to the present invention includes applying a reactive mesogen to said surface and photopolymerizing said reactive mesogen in situ to form the light emitting polymer. The reactive mesogen has the formula: 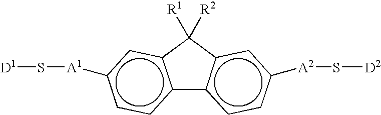
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula:

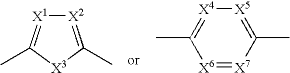

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 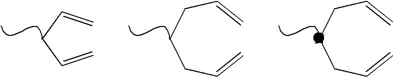
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, CO—S—, —CH═CH—, —C≡C— such that O and S atoms are not directly linked to other O or S atoms. The applying the reactive mesogen to the surface may be by a spin-coating or a solvent casting process. Additionally, the step of applying a copolymer incorporating both linear rod-like hole-transporting and photoreactive side chains to the surface may be included. The above surface may be a photoalignment layer surface. The light emitting polymer may be in the form of a liquid crystal uniaxially aligned by the underlying photoalignment layer surface. The light emitting polymer is in the form of a liquid crystal uniaxially aligned by the liquid crystalline structure of an underlying polymer layer and the underlying polymer may be a charge carrier transport layer.

Another exemplary process for applying a light emitting polymer to a surface according to the present invention includes applying a reactive mesogen to said surface and photopolymerizing said reactive mesogen in situ to form the light emitting polymer. The reactive mesogen mixture comprises two more components having the formula: 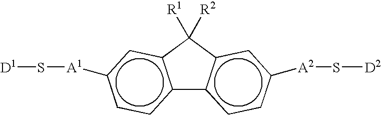
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula:

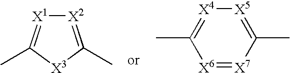

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 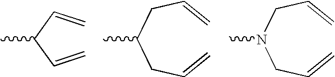
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The mixture may be a thermodynamically stable liquid crystal phase at room temperature. The applying the reactive mesogen to the surface may be by a spin-coating or a solvent casting process. The process may further include applying a copolymer incorporating both linear rod-like hole-transporting and photoreactive side chains to the surface. The surface may be a photoalignment layer. The light emitting polymer may be in the form of a liquid crystal uniaxially aligned by the underlying photoalignment layer surface. The light emitting polymer may be in the form of a liquid crystal uniaxially aligned by the liquid crystalline structure of an underlying polymer layer. The underlying polymer may be a charge carrier transport layer.

Another exemplary process for applying a charge carrier transporting polymer to a surface according to the present invention includes applying a reactive mesogen to said surface and photopolymerizing said reactive mesogen in situ to form the light emitting polymer. The reactive mesogen has the formula: 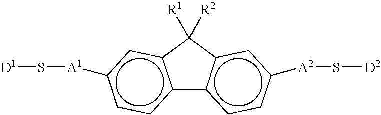
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula:

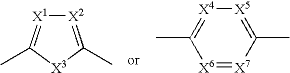

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 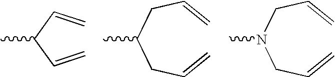
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO, —OCO—, —OCO—O—, —S—CO—, —CO—S—, —CH═CH—, —C≡C— such that O and S atoms are not directly linked to other O or S atoms.

The applying the reactive mesogen to the surface may be done by a spin-coating or solvent casting process. The process may further include applying a copolymer incorporating both linear rod-like hole-transporting and photoreactive side chains to the surface. The surface may be a photoalignment layer. The charge carrier transporting polymer may be in the form of a liquid crystal uniaxially aligned by the underlying photoalignment layer surface. The charge carrier transporting polymer may be in the form of a liquid crystal uniaxially aligned by the liquid crystalline structure of an underlying polymer layer.

Another exemplary process for applying a charge carrier transporting polymer to a surface according to the present invention includes applying a reactive mesogen to said surface and photopolymerizing said reactive mesogen in situ to form the light emitting polymer. The reactive mesogen mixture comprises two more components having the formula: 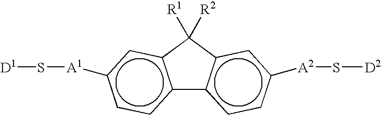
wherein either A¹ or A² consist of a series of two or more aryl biradicals concatenated together in a substantially linear chain connecting the central fluorene unit and flexible spacer units S. Either A¹ and A² or both contain at least two heterocyclic aryl biradicals containing five or six membered aromatic rings with the general formula:

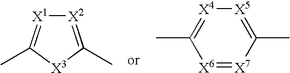

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are spacer groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The D¹ and D² are independently selected from the group consisting of: 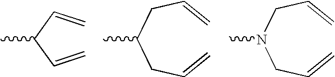
and the R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The mixture may be a thermodynamically stable liquid crystal phase at room temperature. The process may include applying the reactive mesogen to the surface by a spin-coating or a solvent casting process. The process may further include applying a copolymer incorporating both linear rod-like hole-transporting and photoreactive side chains to the surface. The surface may be a photoalignment layer surface. The charge carrier transporting polymer may be in the form of a liquid crystal uniaxially aligned by the underlying photoalignment layer surface. The charge carrier transporting polymer may be in the form of a liquid crystal uniaxially aligned by the liquid crystalline structure of an underlying polymer layer.

Another exemplary compound according to the present invention includes the following structural units: 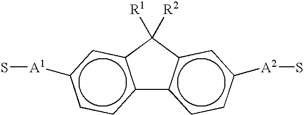
wherein either A¹ or A² consist of a series of two or more aryl biradicals concatenated together in a substantially linear chain connecting the central fluorene unit and flexible tail units S. Either A¹ and A² or both contain at least two heterocyclic aryl biradicals containing five or six membered aromatic rings with the general formula: 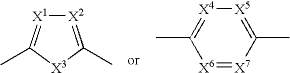
One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are flexible tail groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

Another exemplary process for applying a light emitting layer to a surface according to the present invention includes applying liquid crystalline materials to said surface. The liquid crystalline molecules have the formula: 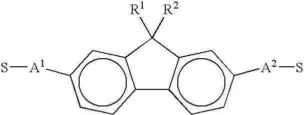
wherein either A¹ or A² consist of a series of two or more aryl biradicals concatenated together in a substantially linear chain connecting the central fluorene unit and flexible tail units S. Either A¹ and A² or both contain at least two heterocyclic aryl biradicals containing five or six membered aromatic rings with the general formula:

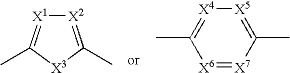

One or more of X¹ and X² are hetero atoms independently selected from N, P, and As, and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S units are flexible tail groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced bye, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The light emitting layer may be a liquid crystal glass. The process may include applying the liquid crystalline material to the surface by a spin-coating or solvent casting process. The process may further include applying a copolymer incorporating both linear rod-like hole-transporting and photoreactive side chains to the surface. The surface may be a photoalignment layer. The light emitting layer may be in the form of a liquid crystal uniaxially aligned by the underlying photoalignment layer surface. The light emitting layer is in the form of a liquid crystal uniaxially aligned by the liquid crystalline structure of an underlying device layer.

Another exemplary process for applying a charge carrier transporting layer to a surface according to the present invention includes applying liquid crystalline materials to said surface. The liquid crystalline molecules have the formula: 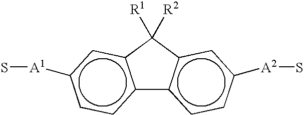
wherein either A¹ or A² consist of a series of two or more aryl biradicals concatenated together in a substantially linear chain connecting the central fluorene unit and flexible tail units S. Either A¹ and A² or both contain at least two heterocyclic aryl biradicals containing five or six membered aromatic rings with the general formula 1.1:

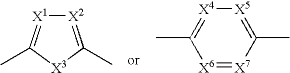

One or more of X¹ and X² are hetero atoms independently selected from N, P, and As, and and X³ may be selected from O, NH, S, PH, Se, AsH, Te, SbH. One or more of X⁴ to X⁷ are independently selected from N, P, and As, and the heterocyclic biradicals may consist of the individual rings pictured above or fused ring systems containing those heterocyclic rings. The S are flexible tail groups independently including branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms. The charge carrier transporting layer may be a liquid crystal glass. The process may include applying the liquid crystalline material to the surface by a spin-coating or a solvent casting process. The process may further include applying a copolymer incorporating both linear rod-like hole-transporting and photoreactive side chains to the surface. The surface may be a photoalignment layer surface. The charge carrier transporting layer may be in the form of a liquid crystal uniaxially aligned by the underlying photoalignment layer surface. The charge carrier transporting layer may be in the form of a liquid crystal uniaxially aligned by the liquid crystalline structure of an underlying device layer.

Another aspect of the present invention is to provide a compound including thienothiophene fused ring structural units combined with the non-conjugated diene and fluorene structural units in the following general formula: B₁—S₁-T₁-(F-T₂)ₚ—F-T₃-S₂—B₂. The B₁ is a non-conjugated diene end group, the B₂ is a non-conjugated diene end group, the F is a fluorene functional unit having the formula: 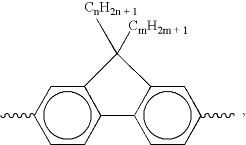
where n is from 1 to 10 and m is from 1 to 10, S₁ and S₂ are spacer units, and at least one of T₁, T₂, and T₃ have the formula: —W—X—Y—. X is selected from the group consisting of: 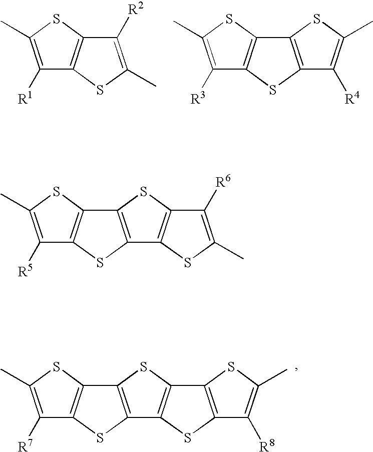
and W and Z are independently selected from the group consisting of: 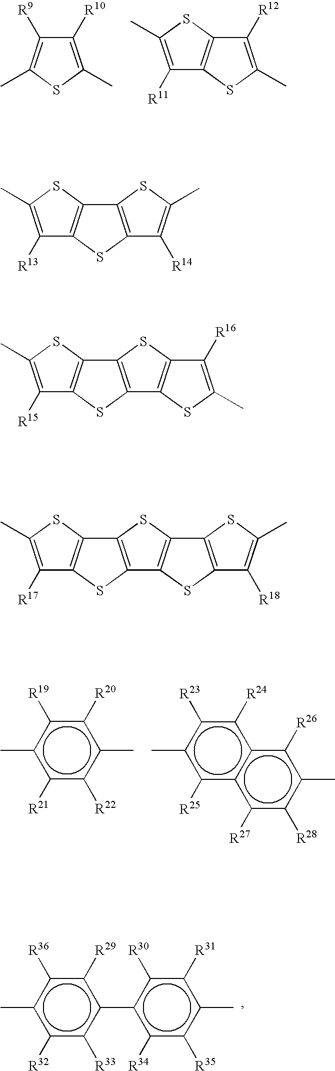
a single bond, and wherein R¹ through R³⁶ are independently selected from the group consisting of H, halogen, CN, NO₂, or branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by A, —S—, —NH—, —NR—, —SiRR—, —CO—, —COO—, —OCO—, —OCO—O—, —S—CO—, —CO—S—, —CH═CH—, —C═C— in such a manner that O and/or S atoms are not directly linked to each other. T₁, T₂, and T₃ that do not have the general formula —W—X—Y— are independently selected from the group consisting of a single bond, 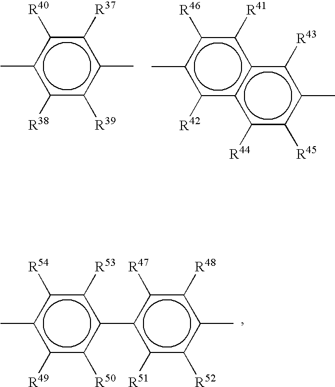
aromatic diradicals and heteroaromatic diradicals wherein R³⁷ through R⁵³ are independently selected from the group consisting of H, halogen, CN, NO₂, and branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms and p=0 to 5.

Another aspect of the present invention is to provide a process for forming a light emitting polymer comprising polymerization of a reactive mesogen having the formula: B₁—S₁-T₁-(F-T₂)ₚ-F-T₃-S₂—B₂. B₁ and B₂ are polymerizable end groups, F is a fluorene functional unit, S₁ and S₂ are spacer units; and T₁, T₂, and T₃ are thienothiophenes units.

Another aspect of the present invention is to provide a polymer including a reactive mesogen having the formula: B₁—S₁-T₁-(F-T₂)ₚ—F-T₃-S₂—B₂. B₁ and B₂ are polymerizable end groups, F is a fluorene functional unit, S₁ and S₂ are spacer units, and T₁, T₂, and T₃ are thienothiophenes units.

Another aspect of the present invention is to provide a polymer including

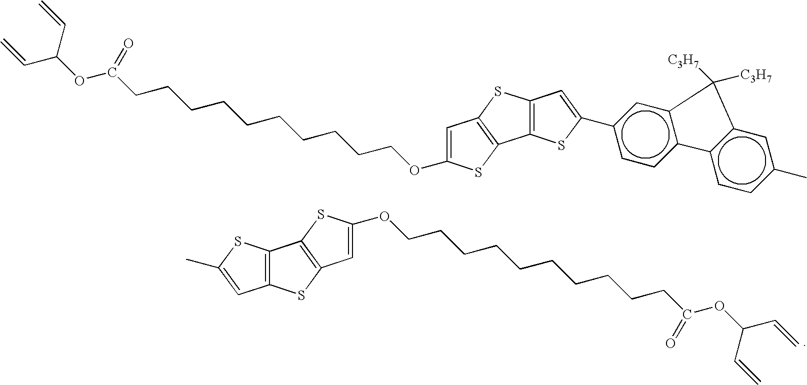

### BRIEF DESCRIPTION OF THE DRAWINGS

The invention will be described in detail with reference to the following drawings in which like reference numerals refer to like elements wherein:

FIG. 1 is a photomicrograph at 73° C. of nematic droplets of the mixture 2 just below the nematic clearing point;

FIG. 2 is a photomicrograph at 25° C. of the nematic Schlieren texture of the mixture 2 just below the nematic clearing point;

FIG. 3 is a differential scanning thermogram as a function of temperature for the first heating and cooling cycle for mixture 2;

FIG. 4 is a cyclic voltammogram of the oxidation of hexa-phenylene 15;

FIG. 5 is an absorbance spectra from a crosslinked network of the symmetrical fluorene diene ester 8 before and after washing in chloroform;

FIG. 6 is a PL spectra of compounds a) 15, b) mixture 2 c) 3 and d) 38;

FIG. 7 illustrates an exemplary structure of an OLED between two electrodes; and

FIG. 8 illustrates the electroluminescence spectrum for Compound 39.

### DETAILED DESCRIPTION

Our previous patent applications have described reactive mesogens with exceptionally low melting points and stable nematic phases that were synthesized containing chromophores that include 9,9-dialkylfluorene structural units. Additionally, reactive mesogens exhibiting room temperature nematic phases were prepared by the appropriate substitution of aliphatic side-chains and end-chains. Alternatively, binary eutectic mixtures of homologous series of compounds were shown to exhibit room temperature nematic phases. Such reactive mesogens may completely suppress the formation of smectic phases. Crosslinking in the nematic phase at room temperature gives completely insoluble thin films. These anisotropic polymer networks may be used as hole-transporting, emission or electron-transporting layers in multilayer OLEDs and may be photolithographically patterned.

The ionization potentials and emission spectra of our other compounds were shown to be modified by incorporating electron-donating and electron-withdrawing groups into the aromatic core of the mesogens. Subsequent work has shown that the ionisation potential of the fluorene containing reactive mesogens may be tuned by chemical modification of the aromatic cores (e.g., a six-ring fluorene may be tuned between about 4.93 to about 5.57 eV) and the emission spectrum may be tuned (e.g., blue to green).

These compounds could be crosslinked to insoluble polymer networks by either thermal or photoinduced generation of free radicals. However, the crosslinking of reactive mesogens to form insoluble polymer networks as charge-transport and/or emission layers in OLEDs often cause a substantial degree of photochemical degradation. Polymer networks formed from reactive mesogens provide a unique and advantageous combination of properties compared to other approaches: they are monodisperse after standard purification procedures; they form insoluble, intractable polymer films by spin coating and subsequent polymerization; these films are photopatternable and some exhibit higher photoluminescence efficiency and improved current-voltage characteristics in prototype OLEDs than the monomers themselves before crosslinking; they may be used to generate polarized emission; the charge-carrier mobility also may exhibit a low field dependence. Photopolymerization, as compared to thermal polymerization, is advantageous because of the pixellation capability and because high temperatures may reduce the order parameter of uniformly oriented reactive mesogens and also lead to photodegradation. The polymerizable end-groups may be polymerized by a radical mechanism in order to avoid the presence of ionic initiator and reaction products within the resultant crosslinked polymer network. These charged ionic contaminants may act as traps and potentially contribute to device failure. An advantage of non-conjugated diene end-groups compared to acrylates or methacrylates is the low tendency of such non-conjugated dienes to polymerize thermally which allows for easier and longer storage. Additionally, the unreacted monomers generally will not polymerize spontaneously during the fabrication operation of an OLED.

The 2,7-disubstituted-9,9-dialkylfluorene group combines a combination of attractive features for light-emitting organic materials. It is the presence of the two alkyl chains at the bridging benzylic position of the 9,9-dialkylfluorene moiety that helps generate the advantageous physical properties associated with these materials. The two alkyl chains give rise to a larger intermolecular distance, which lowers the melting point and increases the solubility in organic solvents compared to the corresponding non-substituted fluorenes. They also contribute to the relatively high viscosity of the 9,9-dialkylfluorenes, which results in a high tendency for glass formation. However, a further advantageous property of the two alkyl chains is their tendency to suppress the formation of smectic phases, whose layered structure induces a much higher viscosity than that of the nematic phase. Thus, the nematic phases more easily macroscopically aligned, e.g., for polarized emission are macroscopically aligned, as compared to the smectic phases. The energy levels of the chromophores may be tailored for hole or electron injection and for blue, green and red emission (and other wavelengths) for full color capability.

Examples of such reactive mesogen materials are shown in tables 1-3.

TABLE 1
Transition temperatures for the symmetrical esters 1-8 and the ethers 9-13.

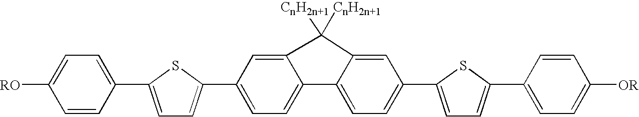

| | | | | | |
|---|---|---|---|---|---|
| n | OR | T_{g} | Cr | N | I |

| | | | | | | | | |
|---|---|---|---|---|---|---|---|---|
| 1 | 3 | OC₃H₆CO₂CH(CH═CH₂)₂ | 62 | • | 92 | • | 116 | • |
| 2 | 3 | OC₄H₈CO₂CH(CH═CH₂)₂ | 45 | • | | • | 120 | • |
| 3 | 3 | OC₅H₁₀CO₂CH(CH═CH₂)₂ | 39 | • | 92 | • | 108 | • |
| 4 | 3 | OC₁₀H₂₀CO₂CH(CH═CH₂)₂ | 18 | • | 92 | (• | 82) | • |
| 5 | 3 | OC₂H₄CH(CH₃)C₂H_{4CO}₂CH(CH═CH₂)₂ | | • | 58 | • | 87 | • |
| 6 | 8 | OC₅H₁₀CO₂CH(CH═CH₂)₂ | −26 | • | 96 | (• | 29) | • |
| 7 | 8 | OC₇H₁₄CO₂CH(CH═CH₂)₂ | −25 | • | 43 | (• | 25) | • |
| 8 | 8 | OC₁₀H₂₀CO₂CH(CH═CH₂)₂ | −27 | • | 41 | (• | 32) | • |
| 9 | 3 | OC₅H₁₀OCH(CH═CH₂)₂ | 25 | • | 101 | • | 116 | • |
| 10 | 3 | OC₆H₁₂OCH(CH═CH₂)₂ | 19 | • | 92 | • | 116 | • |
| 11 | 3 | OC₈H₁₆OCH(CH═CH₂)₂ | 2 | • | 97 | • | 106 | • |
| 12 | 3 | OC₉H₁₈OCH(CH═CH₂)₂ | — | • | 93 | • | 98 | • |
| 13 | 8 | OC₅H₁₀OCH(CH═CH₂)₂ | −25 | • | 97 | (• | 44) | • |

( )Represents a monotropic transition temperature

TABLE 2
Transition temperatures for the hexa-phenylenes 14-23 and the fluoro-substituted hexa-
phenylenes 24-29.

| | | | | | | | |
|---|---|---|---|---|---|---|---|
| X | Y | n | m | T_{g} | Cr | N | I |

| | | | | | | | | | | |
|---|---|---|---|---|---|---|---|---|---|---|
| 14 | H | H | 3 | 5 | | • | 143 | • | 166 | • |
| 15 | H | H | 4 | 5 | 25 | • | 126 | • | 151 | • |
| 16 | H | H | 5 | 5 | | • | 126 | • | 137 | • |
| 17 | H | H | 6 | 5 | | • | 137 | (• | 124) | • |
| 18 | H | H | 8 | 5 | — | • | 91 | • | 109 | • |
| 19 | H | H | 8 | 7 | −26 | • | 52 | • | 103 | • |
| 20 | H | H | 8 | 10 | −20 | • | 38 | • | 96 | • |
| 21 | H | H | 8 | 11 | — | • | 58 | • | 88 | • |
| 22 | H | H | 10 | 7 | — | • | 57 | • | 79 | • |
| 23 | H | H | 10 | 10 | — | • | 53 | • | 88 | • |
| 24 | F | H | 8 | 5 | −16 | • | 93 | (• | 56) | • |
| 25 | F | H | 8 | 7 | — | • | 63 | (• | 52) | • |
| 26 | F | H | 8 | 10 | — | • | 64 | (• | 51) | • |
| 27 | F | H | 8 | 11 | — | • | 70 | (• | 44) | • |
| 28 | H | F | 8 | 10 | −27 | • | 54 | • | 58 | • |
| 29 | H | F | 8 | 11 | −26 | • | 58 | (• | 51) | • |

( )Represents a monotropic transition temperature

TABLE 3
Transition temperatures for the asymmetric reactive mesogen 30-34.

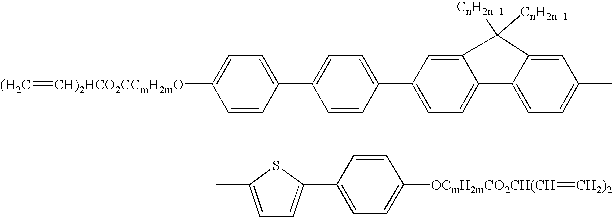

| | | | | | |
|---|---|---|---|---|---|
| n | m | T_{g} | Cr | N | I |

| | | | | | | | | |
|---|---|---|---|---|---|---|---|---|
| 30 | 3 | 5 | 11 | • | 133 | | — | • |
| 31 | 3 | 10 | −2 | • | 44 | • | 113 | • |
| 32 | 6 | 10 | −15 | • | 78 | (• | 75) | • |
| 33 | 8 | 7 | | • | 50 | | — | • |
| 34 | 8 | 10 | | • | −28 | • | 21 | • |

( )Represents a monotropic transition temperature

TABLE 4
Transition temperatures (° C.) for the eight-ring reactive mesogens 35 and 36.

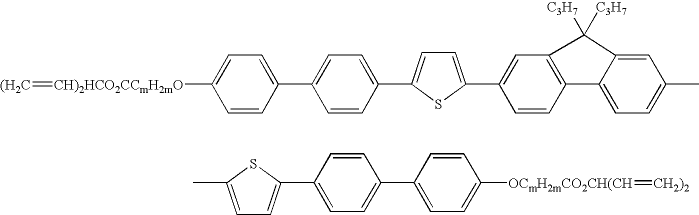

| | m | T_{g} | | Cr | | N | | I |
|---|---|---|---|---|---|---|---|---|
| 35 | 5 | • | — | • | 142 | • | 266 | • |
| 36 10 | • | 72 | • | 134 | • | 228 | • | |

TABLE 5
Transition temperatures for the symmetrical pyrimidine reactive mesogens 37 and 38.

| | n | m | T_{g} | | Cr | | N | | I |
|---|---|---|---|---|---|---|---|---|---|
| 37 | 3 | 5 | • | 20 | • | 128 | (• | 111) | • |
| 38 | 8 | 10 | | | • | 68 | (• | 55) | • |

( ) Represents a monotropic transition temperature

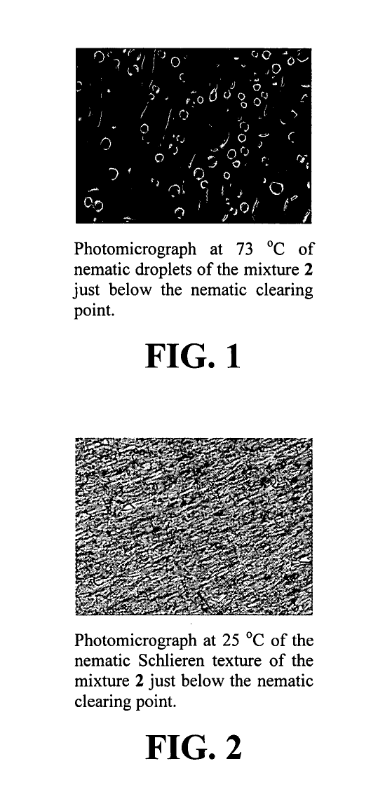

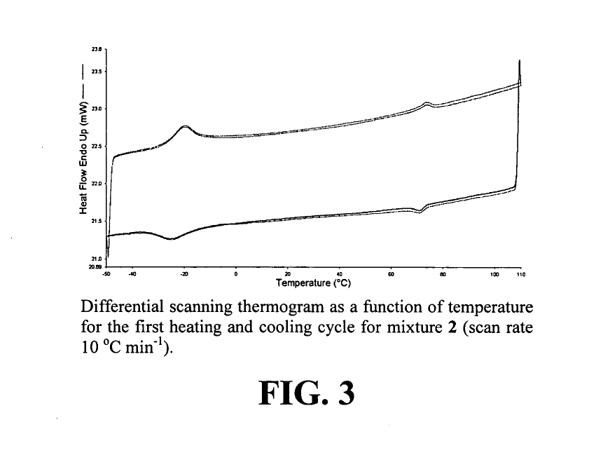

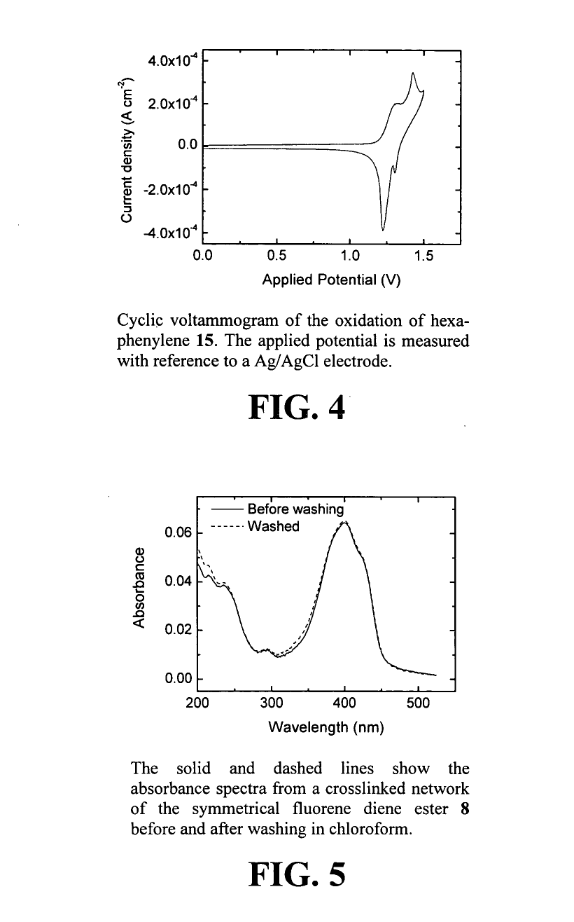

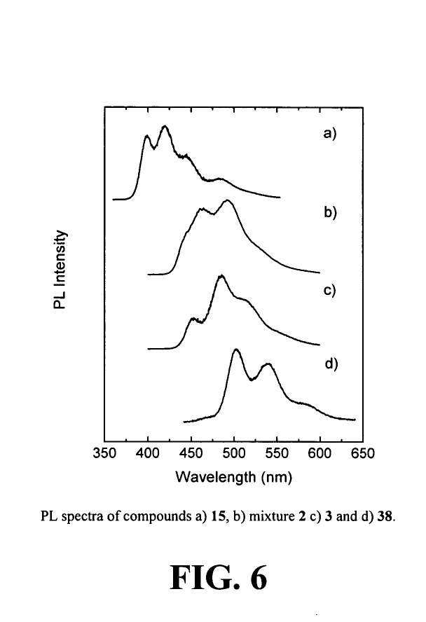

FIG. 1 is a photomicrograph at 73° C. of nematic droplets of the mixture 2 just below the nematic clearing point. Mixture 2 is a 1:1 mixture of the reactive mesogens 31 and 33. FIG. 2 is a photomicrograph at 25° C. of the nematic Schlieren texture of the mixture 2 just below the nematic clearing point. FIG. 3 is a differential scanning thermogram as a function of temperature for the first heating and cooling cycle for mixture 2. FIG. 4 is a cyclic voltammogram of the oxidation of hexa-phenylene 15. FIG. 5 is an absorbance spectrum from a crosslinked network of the symmetrical fluorene diene ester 8 before and after washing in chloroform. FIG. 6 is a PL spectrum of compounds a) 15, b) mixture 2 c) 3 and d) 38.

A problem with the materials of the formulas: 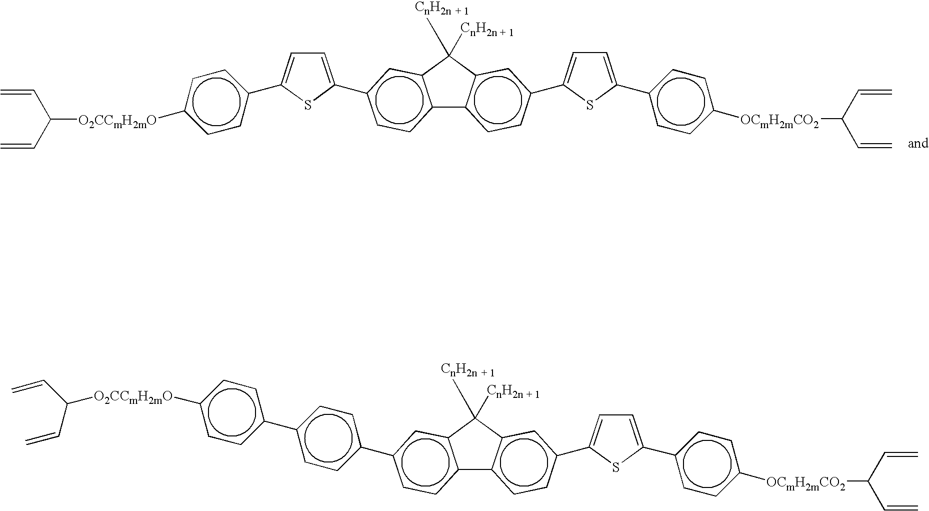
is that the level of current that can be passed through OLED devices produced using them is limited. This may be due to an issue with the efficiency of electron injection into the materials from the OLED cathode. The result of this current limitation is a limitation in output luminance of the OLEDs produced to approximately 200 candelas/m². What is needed are materials similar to the reactive mesogens that have been found to be useful as photocrosslinkable emitter materials such as the following structure: 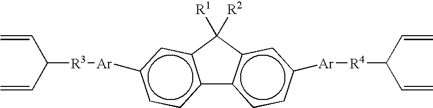
where R¹ and R² are flexible side-chains, most usually alkyl groups and R³ and R⁴ are flexible spacer chains connecting the terminal dienes to the aromatic nucleus of the molecule (R³ and R⁴ are most usually akyleneoxy groups with the oxygen connecting the alkylene chain to the aromatic nucleus), such those described in U.S. patent application Ser. Nos. 10/187,402 and 10/187,381, but that do not have a current carrying limitation when used in OLEDs. U.S. patent application Ser. Nos. 10/187,402 and 10/187,381 are incorporated herein by this reference.

The OLED devices containing emitter layers produced by polymerization of the compound with the formula shown below surprisingly support much higher current levels than the previous devices that are produce by polymerization of fluorene nucleus containing reactive mesogen materials as described above.

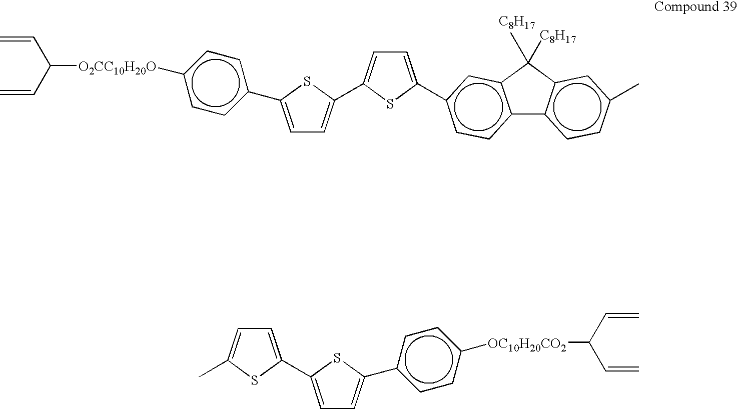

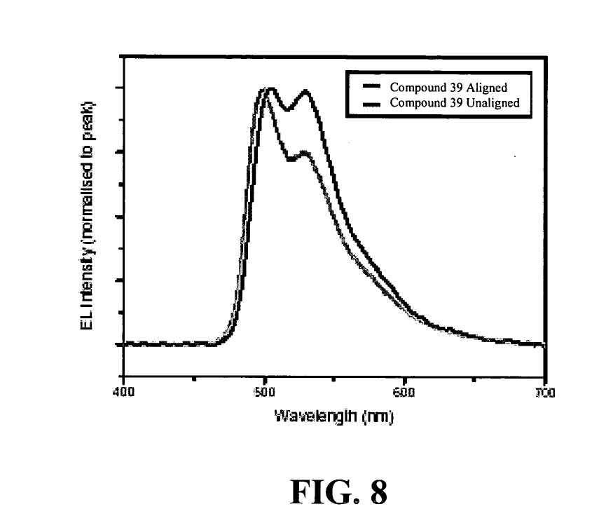

The material, when fabricated into an OLED supports sufficient current flow to yield luminances in excess of 14,000 candelas/m². We believe the more than an order of magnitude increased current is due to the presence of more hetero atoms in the material (four sulfurs in this case) and the concomitant increase in the number of lone pair electrons. The compound above also has a very broad nematic range between 134° C. and 228° C. FIG. 8 illustrates the electroluminescence spectrum for Compound 39.

Other materials with multiple heterocyclic rings in one or both of the Ar radicals adjoining the fluorene nucleus support increased current flow as well. The heterocyclic rings may constitute five or six atoms and may be part of fused ring systems. They may be directly linked together as in compound 39 or non-heterocyclic aromatic ring systems may be inserted between them. The reactive mesogens may include a terminal non-conjugated diene as the polymerizable group. Alternatively, corresponding acrylates and methacrylates may be used.

Further compounds of the present invention include those that combine thienothiophene fused ring structural units with the non-conjugated diene and fluorene structural units in the following general formula:
B₁—S₁-T₁-(F-T₂)ₚ-F-T₃-S₂—B₂ (General Formula 1)
wherein B₁ is a non-conjugated diene end group;
wherein B₂ is a non-conjugated diene end group;
wherein F is the fluorene functional unit has the formula of: 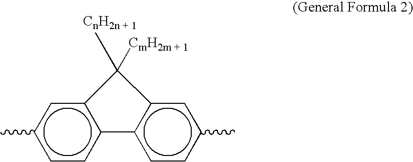
wherein n and m may be from 1 to 10;
wherein S₁ and S₂ are spacer units;
wherein at least one of T₁, T₂, and T₃ may have the formula:
   —W—X—Y— (General Formula 3);
wherein X may be chosen from amongst: 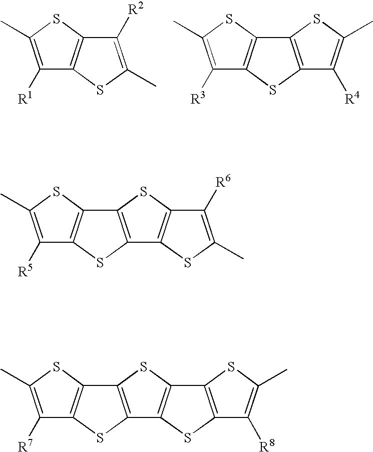
wherein W and Z may be chosen from amongst: 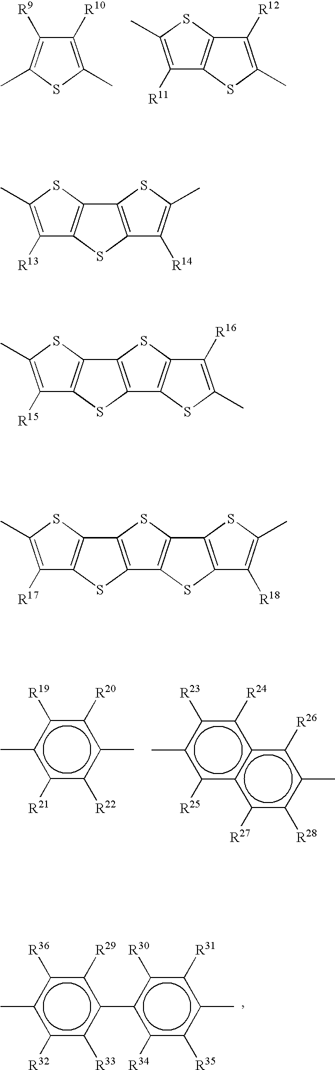
   or a single bond, and wherein R¹ through R³⁶ (if used) may be each independently be chosen from amongst H, halogen, CN, NO₂, or branched, straight chain, or cyclic alkyl groups with 1 to 12 carbon atoms, which are unsubstituted, or mono- or poly-substituted by F, Cl, Br, I, or CN or wherein one or more nonadjacent CH₂ groups may be replaced by —O—, —S—, —NH—, —NR—, —SiRR—, —CO—, —COO—, —OCO—, —OCO—O—, —S—CO—, —CO—S—, —CH═CH—, —C≡C— in such a manner that O and/or S atoms are not directly linked to each other;
wherein the T₁, T₂, and T₃ that do not have the general formula —W—X—Y— may be chosen from amongst a single bond or: 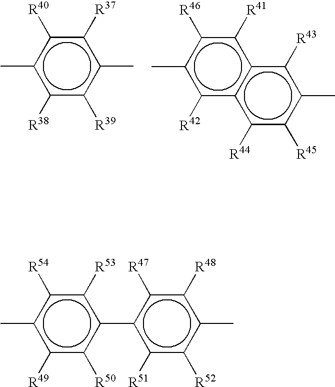
   or other aromatic or heteroaromatic diradicals wherein R³⁷ through R⁵³ (if used) may be each independently H, halogen, CN, NO₂, or branched, straight chain, or cyclic alkyl groups with 1 to 12 carbon atoms, which are unsubstituted, or mono- or poly-substituted by F, Cl, Br, 1, or CN or wherein one or more nonadjacent CH₂ groups may be replaced by —O—, —S—, —NH—, —NR—, —SiRR—, —CO—, —COO—, —OCO—, —OCO—O—, —S—CO—, —CO—S—, —CH═CH—, —C≡C— in such a manner that O and/or S atoms are not directly linked to each other, and
wherein p=0 to 5.

The inclusion of the fluorene in the molecular structures leads to a decrease in the melting points of the reactive mesogens and also appears to stabilize the nematic phase relative to smectic phases.

The non-conjugated diene end group may be chosen from amongst: 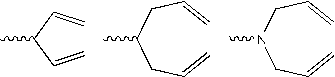
and have the advantage of very little shrinkage or photodegradation on photopolymerization. Of these three end groups, the 1,4-pentadiene end group appears to result in the least shrinkage and photodegradation.

Suitable spacer units (S₁ and S₂) include organic chains such as, for example, flexible aliphatic, amine, ester or ether linkages. The chains may be saturated or unsaturated and may be linear or branched. The presence of spacer groups aids the solubility and further lowers the melting point of the polymer which assists the spin coating thereof.

The compounds and mixtures of the present invention that combine thienothiophene fused ring structural units with the non-conjugated diene and fluorene structural units provide a number of advantageous over the prior art compounds. These compounds and mixtures include room-temperature nematics that may be easily photocrosslinked with a high final degree of polymerization. The layers of crosslinked layers organic semiconductor may be incorporated into electronic devices. Since no initiator is used and since mixtures may be used to form the layers, the resultant device operating lifetimes are uncompromised by the polymerization process.

Liquid Crystalline Behavior.

The replacement of two phenyl rings by thiophene rings and two propyl chains in compound 36 shown in Table 4 by two octyl chains to produce compound 39 shown in Table 8 results in a much lower melting and clearing point. The compound 39 may be supercooled to room temperature and then crosslinked.

TABLE 8
Transition temperatures (° C.) for the eight-ring reactive mesogen 39

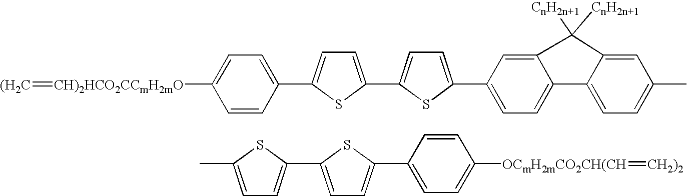

| | M | T_{g} | | Cr | | N | | I |
|---|---|---|---|---|---|---|---|---|
| 39 | 10 | • | 0 | • | 53 | • | 143 | • |

The thermotropic mesophases observed for compound 39 and for our other compounds were investigated between crossed polarizers using optical microscopy. The only phase observed was the nematic phase. Nematic droplets were observed on cooling from the isotropic liquid to form the Schlieren texture with two and four-point brushes characteristic of the nematic phase along with optically extinct homeotropic areas. As a sample is cooled further the texture often formed more optically extinct homeotropic areas, which indicates that the phase is optically uniaxial. The birefringent and homeotropic areas flashed brightly on mechanical disturbance. This behavior and the simultaneous presence of both the homeotropic and the Schlieren texture, confirms that the mesophase observed is indeed a nematic phase.

The values for the transition temperatures were confirmed by differential scanning calorimetry (DSC). Good agreement (≈1-2° C.) with those values determined by optical microscopy were obtained. These values were determined twice on heating and cooling cycles on the same sample. The values obtained on separate samples of the same compounds were reproducible and usually very little thermal degradation was observed even at relatively high temperatures. The base line of the spectra is relatively flat and sharp transition peaks are observed for compound 39 as for our other compounds. The liquid crystalline transition of compound 39 is first order as expected. A degree of supercooling below the melting point was observed on the cooling cycle and compound 39 remained nematic at room temperature for several hours, although its melting point is much higher than room temperature. This may be attributed, at least in part, to the high viscosity of the nematic phase of this material.

Electronic Properties

One advantage of liquid crystal polymer networks is their multilayer capability. Additionally, completely insoluble polymer-network films may be formed from these reactive mesogens. Efficient multilayer OLEDs utilize the matching of energy levels to minimize the barriers for carrier injection and to trap both electron and holes in the luminescent region. The work-function of InSnO is 4.8 eV and that of Ca is 2.9 eV so that hole injection materials with low IPs and electron-injection materials with high EAs are used. The standard strategy to increase/decrease the IP of a molecule is to include electron withdrawing/donating group in its aromatic core. The IP is insensitive to the spacer length of the aliphatic end-chains and side-chains. Table 9 shows the measured IP of compound 39 versus our other compounds.

TABLE 9
The ionization potential and electron affinity of the reactive mesogens 3,
15, 25, 37, 32 and 39.

| | IP^{a} (eV) ± 0.02 | E_{g}^{b} (eV) ± 0.04 | EA^{c} (eV) ± 0.06 | Remark |
|---|---|---|---|---|
| 3 | 5.01 | 2.68 | 2.33 | Reversible |
| 15 | 5.30 | 3.11 | 2.19 | Reversible |
| 25 | 5.36 | 3.10 | 2.26 | Reversible |
| 37 | 5.57 | 3.01 | 2.56 | Irreversible |
| 32 | 5.07 | 2.65 | 2.42 | Reversible |
| 39 | 4.93 | 2.45 | 2.48 | Reversible |

^{a}From CV
^{b}From optical absorption spectrum
^{c}From IP − E_{g}

Compound 39 has the lowest ionization potential, 4.93 eV and is therefore suitable as a hole injection/luminescent material in a three layer OLED. However, the somewhat lower IP as compared to compound 38 does not explain the extremely large increase in current carrying capacity and consequent greatly increased device luminance. We attribute this to the increased current carrying capacity of the material.

The ionization potentials of the reactive mesogens may be measured electrochemically by cyclic voltammetry using a computer-controlled scanning potentiostat (Solartron 1285). 1 mM of the compound was dissolved in 5 cm⁻³ of an electrolytic solution of 0.1M tetrabutylammonium hexafluorophosphate in dichloromethane. The solution was placed in a standard three-electrode electrochemical cell. A glassy carbon electrode was used as the working electrode. Silver/silver chloride (3M NaCl and saturated Ag/Cl)) and a platinum wire formed the reference and counter electrodes respectively. The electrolyte was recrystallized twice before use and oxygen contamination was avoided by purging the solution with dry Argon before each measurement. The measured potentials were corrected to an internal ferrocene reference added at the end of each measurement. A typical scan rate of 20 mV s⁻¹ was used. Two scans were performed to check the repeatability. FIG. 4 is such a cyclic voltammogram of the oxidation of hexa-phenylene 15.

The onset potential for oxidation, Eₒₓ is defined by a step change in current and is obtained from the intersection of the two tangents at the current discontinuity based on the empirical relationship proposed by Bredas, IP=[Eₒₓ+4.4] eV. The EA may be estimated by subtraction of the optical bandedge, taken as the energy of the onset of absorption of the compound, from the IP. However, this approximation does not include a correction for the exciton binding energy. Thin films of the materials were prepared by spin coating from a 0.5-2.0% weight solution in chloroform onto quartz substrates. All the processing was carried out in a glove box filled with dry nitrogen to avoid oxygen and moisture contamination. The photopolymerizable films were polymerized in a nitrogen-filled chamber using UV light from a Helium Cadmium laser at 325 nm with a constant intensity of 50 mW cm⁻². PL and EL were measured with the samples mounted in a chamber filled with dry nitrogen using a photodiode array (Ocean Optics S2000) with a spectral range from 200 nm to 850 nm and a resolution of 2 nm.

Synthetic pathways for materials should be as short as possible to facilitate commercialization, such as the exemplary synthetic pathway shown below:

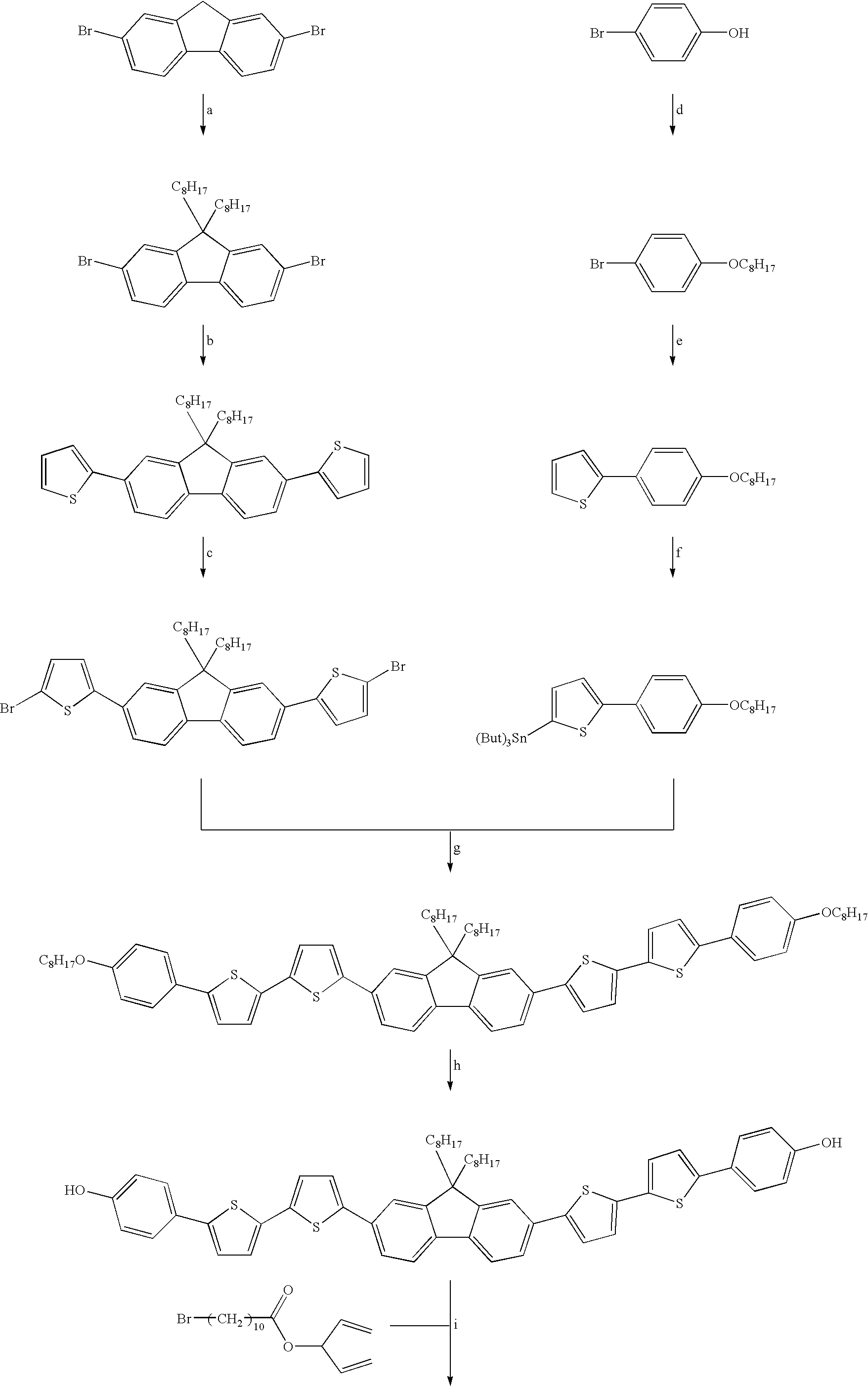

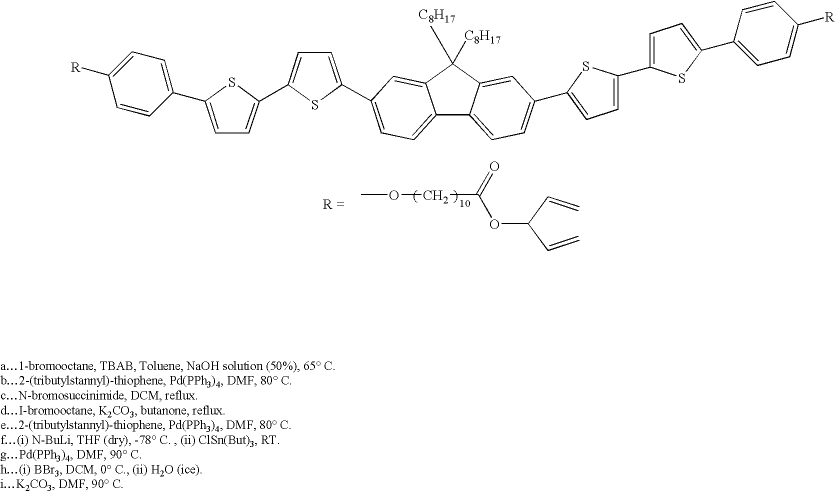

Methodology

Other materials with multiple heterocyclic rings in one or both of the Ar radicals should support increased current flow as well. The heterocyclic rings may constitute five or six atoms and may be part of fused ring systems. They may be directly linked together as in the above compound or non-heterocyclic aromatic ring systems may be inserted between them. For example, the following compounds should support increased current flow in OLED devices.

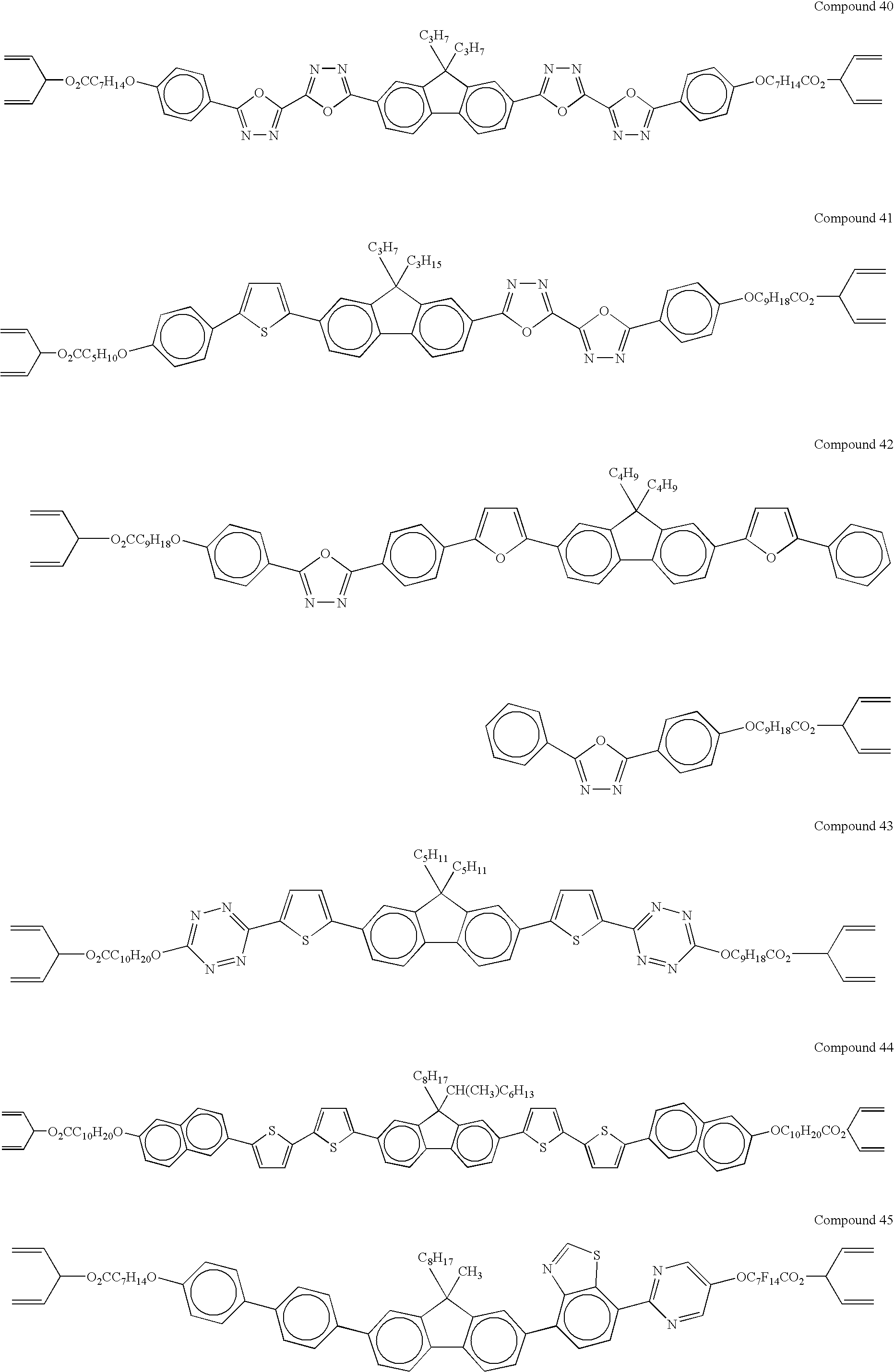

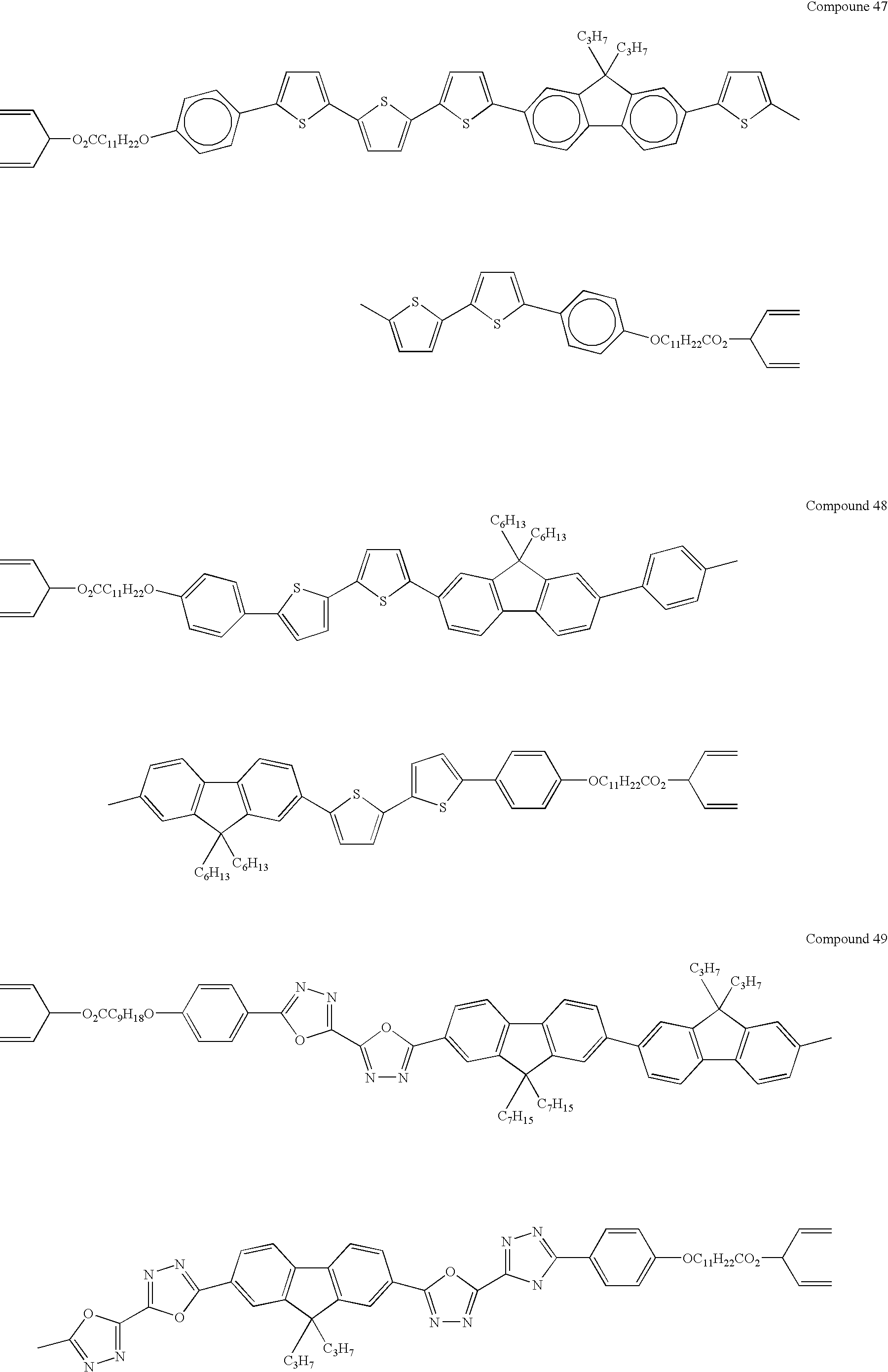

A synthetic scheme for compound 40 is as follows:

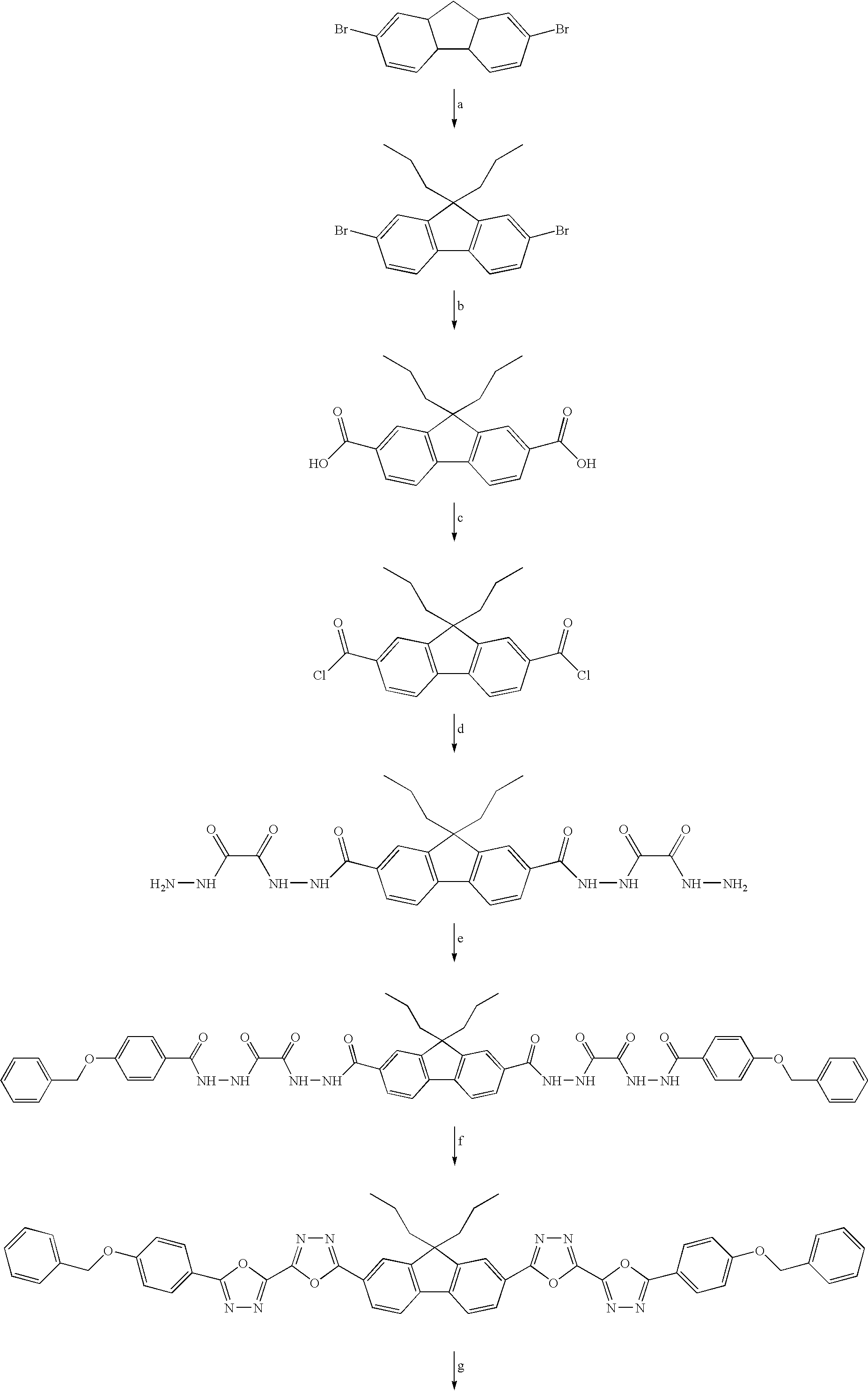

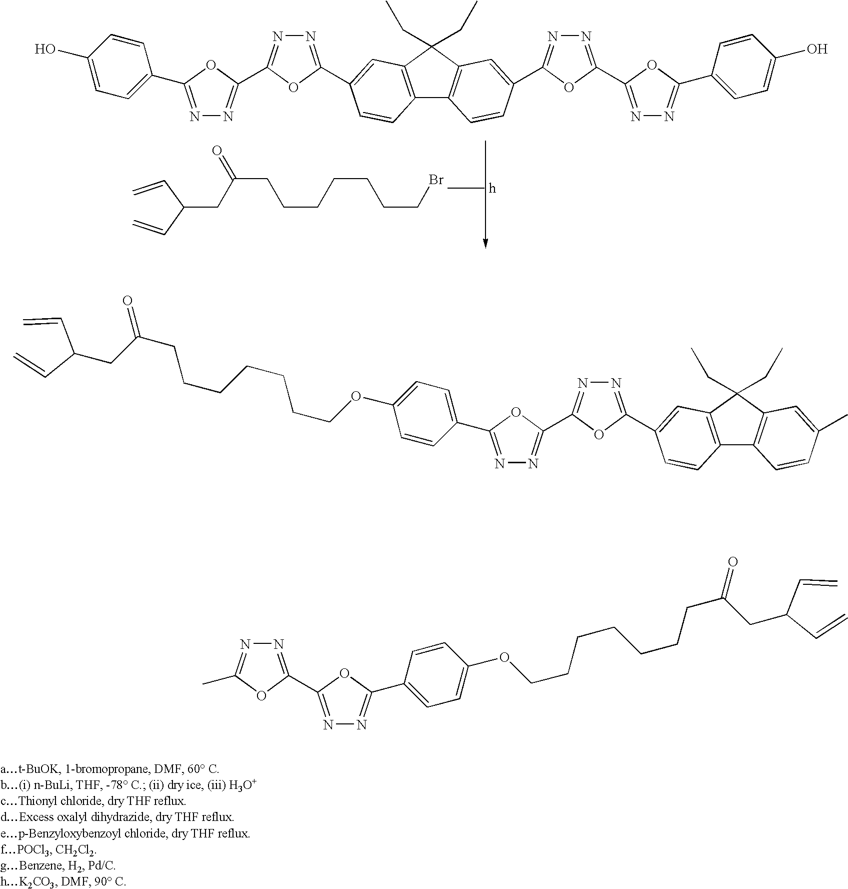

Compound 50 has the following formula: 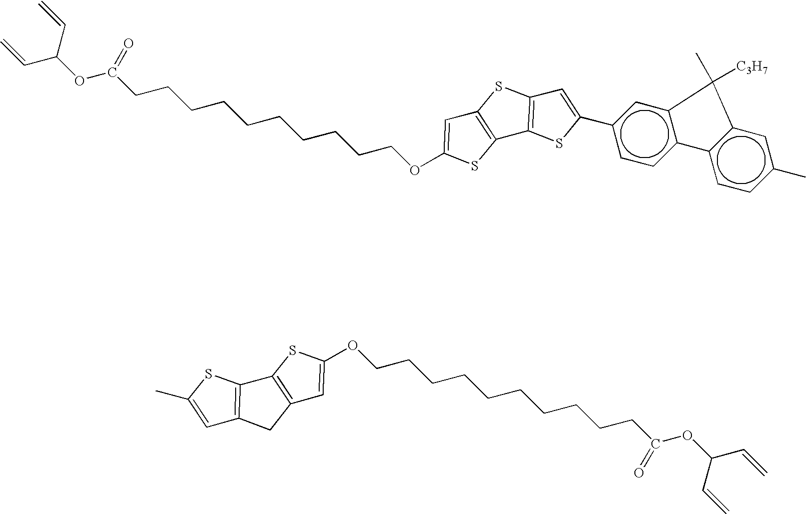
is another exemplary example of the compounds that may be prepared according to the present invention. Compound 50 may be synthesized by the following steps:

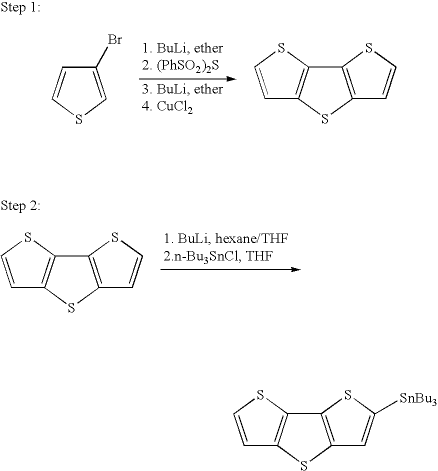

Additional explanation of steps 1 and 2 may be found in published US Patent Application No. 2003/0080322, which is incorporated herein by reference.

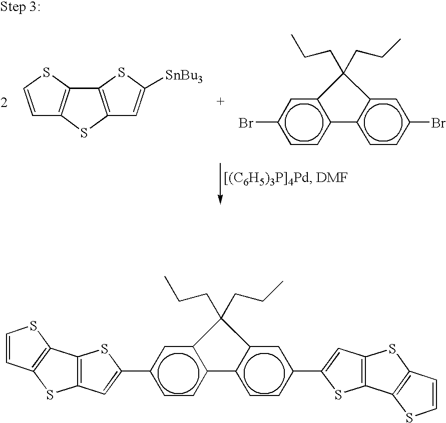

Step 3 is similar to the Stille arylation using 2-(tributylstannyl)thiophene similar to the Stille arylation using 2-(tributylstannyl)thiophene carried out in published US Patent Application No. 2003/0119936, which is incorporated herein by reference.

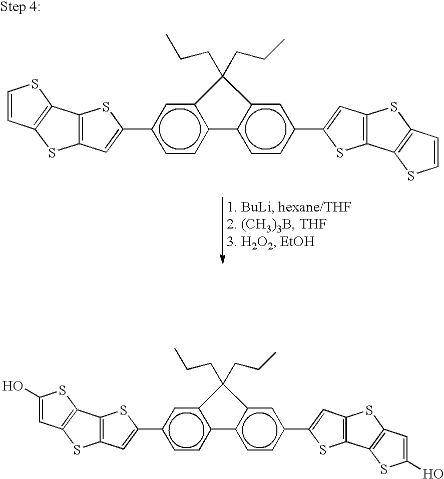

Further explanation of step 4 may be found in M. F. Hawthornr, J. Org. Chem 22, 1001 (1957), which is incorporated herein by reference.

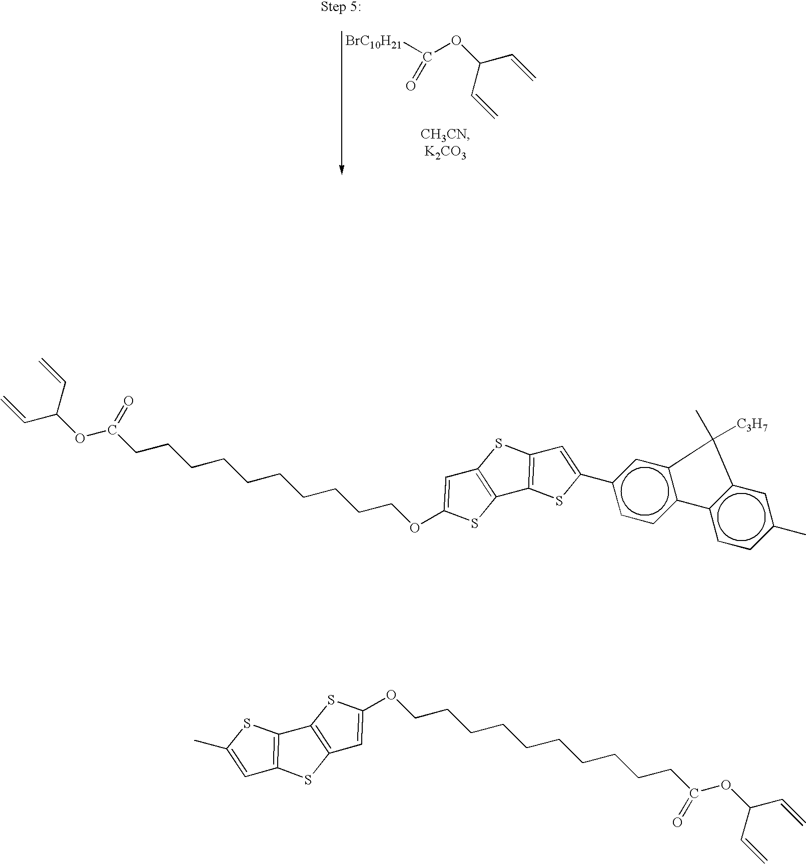

Step 5 is similar to the Williamson reaction run in U.S. Patent Application 2003/0119936, which is incorporated herein by reference.

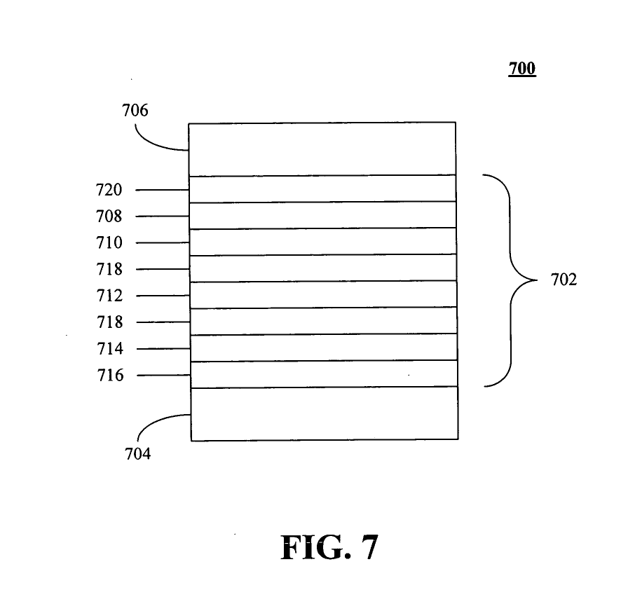

FIG. 7 illustrates an exemplary structure OLED device 700 utilizing the materials described above, including an OLED emitter layer 702 between two electrodes 704, 706. This OLED emitter layer 702 includes a hole injection layer 708, hole transport layer 710, an emitter 712, an electron transport layer 714, an electron injection layer 716, and charge carrier blocker layers 718. The layers of the OLED emitter layer 702 may be produced one layer at a time any may be made from any suitable materials including those discussed herein. In addition to the materials disclosed herein, other materials may be found in, for example, U.S. patent application Ser. Nos. 10/187,381, 10/187,402 and 10/187,396 which were respectively published as 2003/0119936, 2003/0099862 and 2003/0099785, respectively, describe certain exemplary materials that may be used to from the OLED emitter layer 702. These three published applications are hereby incorporated herein by reference. The three published applications each disclose liquid crystalline materials that may be aligned and combined with other layers in the OLED emitter layer 702 which also may have aligned liquid crystalline order. The alignment of one of the layers of the OLED emitter layer 702 may result in subsequently formed layers with liquid crystal properties also being aligned. Such devices having aligned layers may be fabricated on a suitable alignment layer 720 and may include other elements not shown. Alternatively, some of these layers (including the alignment layer 720) may be omitted, a subset of adjacent layers may be built up according to this method, or subset of adjacent layers may be built up according to this method with some of the layers (including the alignment layer) being omitted.

The materials disclosed herein as well as the materials disclosed in U.S. patent application Ser. Nos. 10/187,381, 10/187,402 and 10/187,396, any other suitable alignable material, or any suitable unalignable material may be deposited and then crosslinked to form a crosslinked polymer network. By using a mixture of polymerizable (crosslinkable) materials instead of a single polymerizable material, the rate of polymerization may be increased. This increased polymerization rate facilitates room temperature fabrication in much shorter times and with much less energy being applied. This decrease in the energy being applied into the organic material decreases the amount of degradation produced by the polymerization process. Additionally, the use of a mixture may also improve the crosslinking density, may improve the quality or uniformity of alignment for alignable materials, and may improve the uniformity of the crosslinked polymer network.

As an example, compound 39 may mixed with a mixture of compounds 7 and 8 in a ratio of 60:20:20 to produce a low melting nematic mixture that has superior current carrying capacity as compared to compounds 7 and 8. Since compounds 7 and 8 have a larger HOMO to LUMO energy band gap than does compound 39, exciton energy that may be produced in molecules of compounds 7 and 8 is transferred to compound 39, so that the emission spectrum of the composite material is that of compound 39.

Solvent solutions of binary or other mixtures of charge-transporting and/or light-emitting reactive mesogens with liquid crystalline phases (e.g., nematic or smectic phases) may be spin coated on a conducting photoalignment layer. The spin coating may be done at room temperature to form a film of liquid crystal either in a liquid crystalline phase that is thermodynamically stable at room temperature or in a supercooled liquid crystalline phase below its normal solid to liquid crystal phase transition temperature. Mixtures with thermodynamically stable liquid crystalline phases at room temperature have the advantage of lower viscosity and subsequent ease of crosslinking polymerization. The photoalignment layer aligns the reactive mesogen mixtures at room temperature on the substrate surface with the liquid crystalline director in the plane of the substrate such that one or more monodomains with planar orientation is formed. The charge injection and transport in the crosslinked polymer network is facilitated by the planar orientation. The presence of many different domains does not impair the charge injection and transport of the layers or the emission properties of devices containing such layers. The photoalignment layer may be irradiated by plane polarized UV light to create uniformly anisotropic surface energy at the layer surface. When the reactive mesogen mixture is subsequently coated on the photoalignment layer, the mixture and subsequent polymer network produced on crosslinking have a macroscopic monodomain. Additionally, the polymer network is insoluble and intractable which allows further layers with a different function to be deposited subsequently in a similar fashion.

The photoalignment layer may be used to align a layer of a reactive mesogen of the invention or a mixture of reactive mesogens that includes one or more reactive mesogens of the invention that are solvent cast on the photoalignment layer. The aligned reactive mesogen becomes a polymeric hole transport layer with liquid crystalline order after crosslinking by exposure to UV radiation. Then a second layer of a mixture of reactive mesogens may be solvent cast on top of the hole transport layer. This second layer is aligned into a liquid crystalline monodomain by interaction with the aligned surface of the hole transport layer. The alignment of the second layer is believed to be achieved by molecular interactions between the molecules of the reactive mesogen materials at the interface between the two layers. The second reactive mesogen monolayer may now be crosslinked by exposure to UV radiation to form a polymeric emitter layer. Thus a series of organic semiconductor layers with liquid crystalline order may be built up with all of the molecular cores of the polymers oriented in the same direction.

If the polymerization process does not need an initiator, such as a photoinitiator, there will be no unreacted initiators to quench emission or degrade the performance and lifetime. For example, ionic photoinitiators may act as impurities in finished electronic devices and degrade the performance and lifetime of the devices.

If included, any suitable conducting photoalignment layer may be used. For example, the photoalignment layers described in published U.S. application 2003/0021913 may be used. Alternatively, alignment may be achieved by any other suitable alignment layer or may be achieved without an alignment layer (e.g., the application of electric or magnetic fields, the application of thermal gradients or shear, surface topology, another suitable alignment technique or the combination of two or more techniques). However, rubbed alignment layers are not suitable for organic semiconductor layers and elements, such as the emitter layer in an organic light emitting device or semiconductor layers in integrated circuitry, because the organic layers and elements in such devices are thinner than the amplitude of the surface striations produced in alignment layers by rubbing. In some cases, the roughness resulting from the rubbing process has a thickness on the order of the thickness of the organic layers and elements. Additionally, diverse alignments may be imparted by an alignment layer(s) or technique(s). These diverse alignments may be in a pattern suitable for use in a pixelated device.

The crosslinking density of a network formed from a mixture of polymerizable monomers is higher than that of a network formed by the polymerization of the corresponding individual monomers. The increased crosslinking density may result because in formulating a mixture the solid to liquid crystal transition temperature is depressed below that of any of the individual components and may be depressed below room temperature. This means that the mixture has a thermodynamically stable liquid crystalline phase at room temperature and, as a result, has considerably reduced viscosity as compared to the supercooled glassy liquid crystalline phases of the individual components. This in turn means that reactive mesogen molecules are more mobile within the room temperature phase and thus are able to more quickly and more easily orient themselves to initiate the crosslinking reactions. Such anisotropic polymer network having a higher crosslinking density improves the performance of devices including layers, films or elements fabricated from the network and results in more stable devices.

Although several embodiments of the present invention and its advantages have been described in detail, it should be understood that changes, substitutions, transformations, modifications, variations, permutations and alterations may be made therein without departing from the teachings of the present invention, the spirit and the scope of the invention being set forth by the appended claims.

1. A compound comprising:
the following structural units: 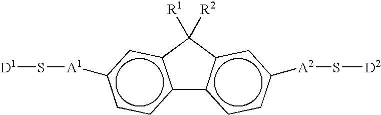
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 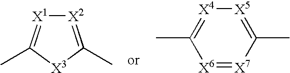
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms;
and wherein D¹ and D² are independently selected from the group consisting of: 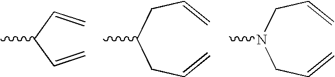
and wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

2. A process for forming a light emitting polymer comprising photopolymerization of a reactive mesogen having the formula: 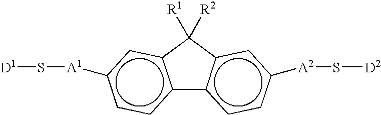
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 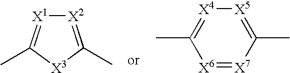
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 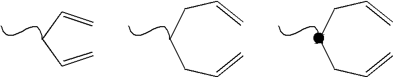
and
wherein R¹ and R² independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

3. A light emitting polymer made by the process of claim 2, wherein the polymer is a liquid crystal.

4. A light emitting polymer according to claim 3, wherein the polymer is aligned to emit polarized light.

5. A process for forming a light emitting polymer comprising photopolymerization of a reactive mesogen mixture composed of two more components, at least one of the two more components having the formula: 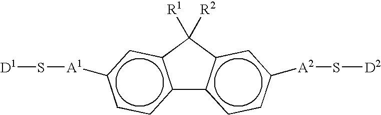
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 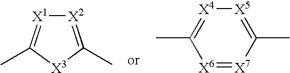
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 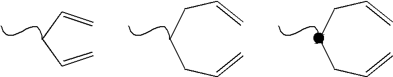
and
wherein R¹ and R² independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

6. The process of claim 5, wherein the mixture has a thermodynamically stable liquid crystal phase at room temperature.

7. A process for forming a polymeric charge carrier transport layer comprising photopolymerization of a reactive mesogen having the formula: 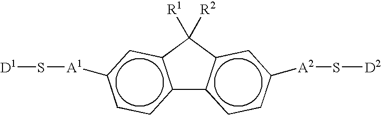
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 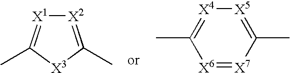
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 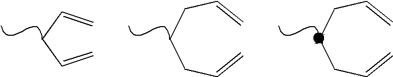
and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —O—, —COO—, —OCO—, —OCO—O—, —S—CO—, —CO—S—, —CH═CH—, —C≡C— such that O and S atoms are not directly linked to other O or S atoms.

8. A process for forming a polymeric charge carrier transport layer comprising photopolymerization of a reactive mesogen mixture composed of two or more components, at least one of the two or more components having the formula: 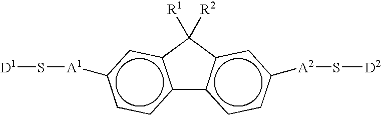
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 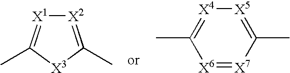
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 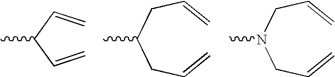
and wherein R¹ and R² independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

9. The process of claim 8 wherein the mixture has a thermodynamically stable liquid crystal phase at room temperature.

10. A process for applying a light emitting polymer to a surface comprising
applying a reactive mesogen to a surface: and
photopolymerizing the reactive mesogen in situ to form the light emitting polymer,
wherein the reactive mesogen has the formula: 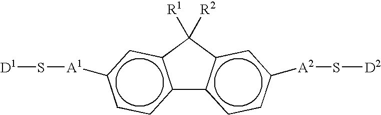
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 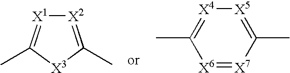
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 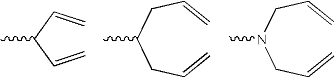
and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO, —OCO—, —OCO—O—, —S—CO—, —CO—S—, —CH═CH—, —C≡C— such that O and S atoms are not directly linked to other O or S atoms.

11. A process according to claim 10, further comprising applying the reactive mesogen to the surface by a spin-coating or other solvent casting process.

12. A process according to claim 10, further comprising applying a copolymer incorporating both linear rod-like hole-transporting and photoreactive side chains to the surface.

13. A process according to claim 10, wherein the surface is a photoalignment layer.

14. A process according to claim 10, wherein the light emitting polymer is a liquid crystal uniaxially aligned by the underlying photoalignment layer surface.

15. A process according to claim 10, wherein the light emitting polymer is a liquid crystal uniaxially aligned by the liquid crystalline structure of an underlying polymer layer.

16. The process according to claim 15, wherein the underlying polymer is a charge carrier transport layer.

17. A process for applying a light emitting polymer to a surface comprising:
applying a reactive mesogen to a surface; and
photopolymerizing said reactive mesogen in situ to form the light emitting polymer,
wherein the reactive mesogen comprises two or more components, at least one of the two or more components having the formula: 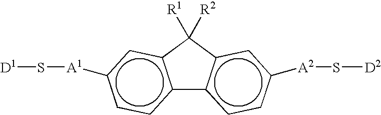
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 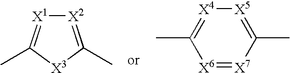
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 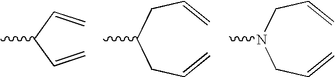
and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

18. A process according to claim 17, wherein the reactive mesogen has a thermodynamically stable liquid crystal phase at room temperature.

19. A process according to claim 17, further comprising applying the reactive mesogen to the surface by a spin-coating or other solvent casting process.

20. A process according to claim 17, further comprising applying a copolymer incorporating both linear rod-like hole-transporting and photoreactive side chains to the surface.

21. A process according to claim 17, wherein the surface is a photoalignment layer.

22. A process according to claim 17, wherein the light emitting polymer is a liquid crystal uniaxially aligned by the underlying photoalignment layer surface.

23. A process according to claim 17, wherein the light emitting polymer is a liquid crystal uniaxially aligned by the liquid crystalline structure of an underlying polymer layer.

24. The process according to claim 23, wherein the underlying polymer is a charge carrier transport layer.

25. A process for applying a charge carrier transporting polymer to a surface comprising
applying a reactive mesogen to a surface: and
photopolymerizing said reactive mesogen in situ to form the light emitting polymer,
wherein the reactive mesogen has the formula: 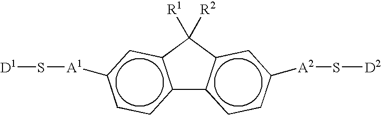
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 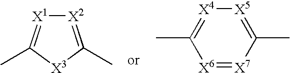
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 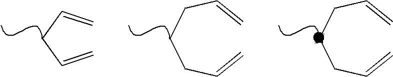
and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

26. A process according to claim 25, comprising applying the reactive mesogen to the surface by a spin-coating or other solvent casting process.

27. A process according to claim 25, further comprising applying a copolymer incorporating both linear rod-like hole-transporting and photoreactive side chains to the surface.

28. A process according to claim 25, wherein the surface is a photoalignment layer.

29. A process according to claim 25, wherein the charge carrier transporting polymer is a liquid crystal uniaxially aligned by the underlying photoalignment layer surface.

30. A process according to claim 25, wherein the charge carrier transporting polymer is in the form of a liquid crystal uniaxially aligned by the liquid crystalline structure of an underlying polymer layer.

31. A process for applying a charge carrier transporting polymer to a surface comprising:
applying a reactive mesogen to a surface; and
photopolymerizing said reactive mesogen in situ to form the light emitting polymer,
wherein the reactive mesogen mixture comprises two or more components, at least one of the two or more components having the formula: 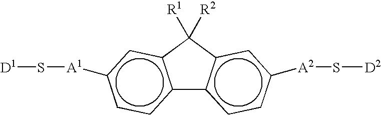
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible spacer units S, and
wherein in either A¹ and A² or both contain at least two heterocyclic aryl biradicals containing five or six membered aromatic rings with the general formula: 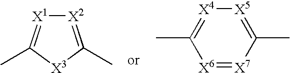
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 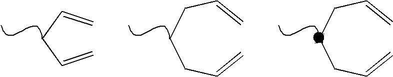
and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

32. A process according to claim 31, wherein the reactive mesogen has a thermodynamically stable liquid crystal phase at room temperature.

33. A process according to claim 31, comprising applying the reactive mesogen to the surface by a spin-coating or other solvent casting process.

34. A process according to claim 31, further comprising applying a copolymer incorporating both linear rod-like hole-transporting and photoreactive side chains to the surface.

35. A process according to claim 31, wherein the surface is a photoalignment layer.

36. A process according to claim 31, wherein the charge carrier transporting polymer is a liquid crystal uniaxially aligned by the underlying photoalignment layer surface.

37. A process according to claim 31, wherein the charge carrier transporting polymer is a liquid crystal uniaxially aligned by the liquid crystalline structure of an underlying polymer layer.

38. A compound comprising:
the following structural units: 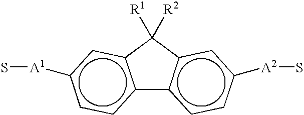
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible tail units S, and
wherein in either A¹ and A² or both contain at least two heterocyclic aryl biradicals containing five or six membered aromatic rings with the general formula: 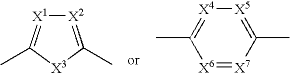
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are flexible tail groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO, —OCO—, —OCO—O—, —S—CO—, —CO—S—, —CH═CH—, —C≡C— such that O and S atoms are not directly linked to other O or S atoms.

39. A process for applying a light emitting layer to a surface comprising:
applying liquid crystalline molecules to a surface;
wherein the liquid crystalline molecules have the formula: 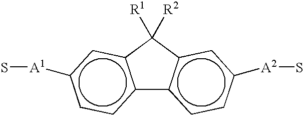
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible tail units S, and
wherein in either A¹ and A² or both contain at least two heterocyclic aryl biradicals containing five or six membered aromatic rings with the general formula: 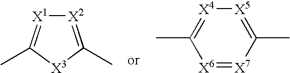
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are flexible tail groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms.

40. The process of claim 39 wherein the light emitting layer is a liquid crystal glass.

41. A process according to claim 39, comprising applying the liquid crystalline molecules to the surface by a spin-coating or other solvent casting process.

42. A process according to claim 39, further comprising applying a copolymer incorporating both linear rod-like hole-transporting and photoreactive side chains to the surface.

43. A process according to claim 39, wherein the surface is a photoalignment layer.

44. A process according to claim 39, wherein the light emitting layer is a liquid crystal uniaxially aligned by the underlying photoalignment layer surface.

45. A process according to claim 39, wherein the light emitting layer is a liquid crystal uniaxially aligned by the liquid crystalline structure of an underlying device layer.

46. A process for applying a charge carrier transporting layer to a surface comprising
applying liquid crystalline materials to the surface;
wherein the liquid crystalline molecules have the formula: 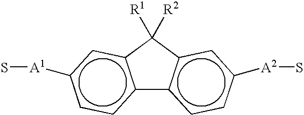
wherein A¹ and A² are selected from a single bond, an aryl biradical, or a series of two or more aryl biradicals concatenated together in a substantially linear chain connecting the central fluorene unit and flexible spacer units S, and
wherein at least one of A¹ and A² comprise a series of two or more aryl biradicals concatenated together in a substantially linear chain connecting the central fluorene unit and flexible tail units S, and
wherein in either A¹ and A² or both contain at least two heterocyclic aryl biradicals containing five or six membered aromatic rings with the general formula: 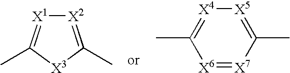
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are flexible tail groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹ and R² independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, CH═CH—, —C≡C— such that O and S atoms are not directly linked to other O or S atoms.

47. The process of claim 46 wherein the charge carrier transporting layer is a liquid crystal glass.

48. A process according to claim 46, comprising applying the liquid crystalline material to the surface by a spin-coating or other solvent casting process.

49. A process according to claim 46, further comprising applying a copolymer incorporating both linear rod-like hole-transporting and photoreactive side chains to the surface.

50. A process according to claim 46, wherein the surface is a photoalignment layer.

51. A process according to claim 46, wherein the charge carrier transporting layer is a liquid crystal uniaxially aligned by the underlying photoalignment layer surface.

52. A process according to claim 46, wherein the charge carrier transporting layer is a liquid crystal uniaxially aligned by the liquid crystalline structure of an underlying device layer.

53. A compound comprising:
the following structural units: 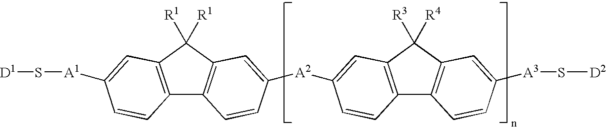
wherein A¹ and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 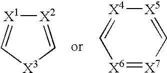
wherein one or more of X¹ and X² are independently selected from, but not limited to N, P, and As, and
wherein X³ may be selected from O, NH, S, PH, Se, AsH, Te, SbH, and
wherein one or more of X⁴ to X⁷ are independently selected from N, P, and As, and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 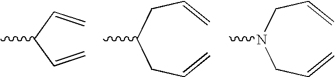
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, CH═CH—, —C≡C— such that O and S atoms are not directly linked to other O or S atoms, and
wherein n=1 to 4.

54. A process for forming a light emitting polymer comprising photopolymerization of a reactive mesogen having the formula: 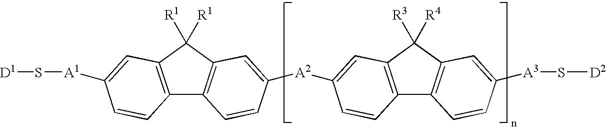
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 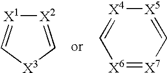
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 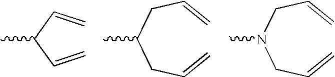
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

55. A light emitting polymer made by the process of claim 54, wherein the polymer is a liquid crystal.

56. A light emitting polymer according to claim 54, wherein the polymer is aligned to emit polarized light.

57. A process for forming a light emitting polymer comprising photopolymerization of a reactive mesogen mixture composed of two more components at least one of which having the formula: 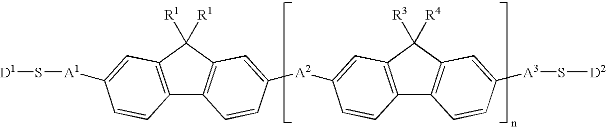
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 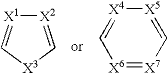
wherein one or more of X¹ and X² are independently selected from, but not limited to N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, CO—S—, —CH═CH—, —C≡C— such that O and S atoms are not directly linked to other O or S atoms; and
wherein D¹ and D² are independently selected from the group consisting of: 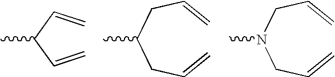
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

58. The process of claim 57, wherein the mixture has a thermodynamically stable liquid crystal phase at room temperature.

59. A process for forming a polymeric charge carrier transport layer comprising photopolymerization of a reactive mesogen having the formula: 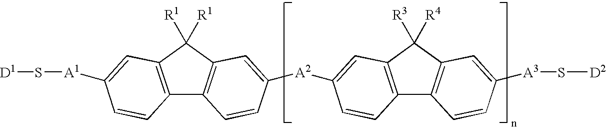
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 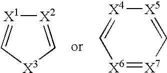
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 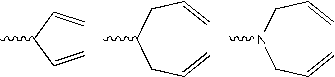
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

60. A process for forming a polymeric charge carrier transport layer comprising photopolymerization of a reactive mesogen mixture composed of two or more components, at least one of the two or more components having the formula: 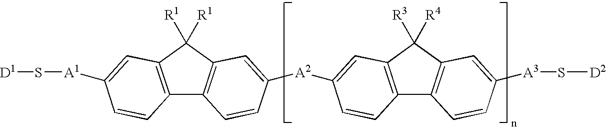
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 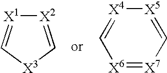
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 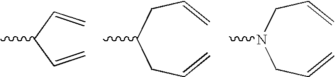
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

61. The process of claim 60 wherein the mixture has a thermodynamically stable liquid crystal phase at room temperature.

62. A process for applying a light emitting polymer to a surface comprising
applying a reactive mesogen to a surface: and
photopolymerizing the reactive mesogen in situ to form the light emitting polymer,
wherein the reactive mesogen has the formula: 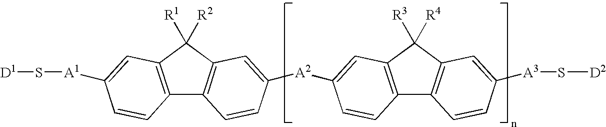
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 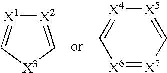
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 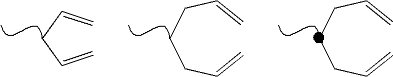
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

62. A process according to claim 61, further comprising applying the reactive mesogen to the surface by a spin-coating or other solvent casting process.

63. A process according to claim 61, further comprising applying a copolymer incorporating both linear rod-like hole-transporting and photoreactive side chains to the surface.

64. A process according to claim 61, wherein the surface is a photoalignment layer.

65. A process according to claim 61, wherein the light emitting polymer is a liquid crystal uniaxially aligned by the underlying photoalignment layer surface.

66. A process according to claim 61, wherein the light emitting polymer is a liquid crystal uniaxially aligned by the liquid crystalline structure of an underlying polymer layer.

67. The process according to claim 66, wherein the underlying polymer is a charge carrier transport layer.

68. A process for applying a light emitting polymer to a surface comprising:
applying a reactive mesogen to a surface; and
photopolymerizing said reactive mesogen in situ to form the light emitting polymer,
wherein the reactive mesogen comprises two more components at least one of which having the formula: 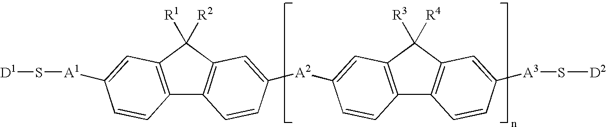
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 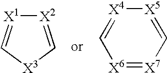
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 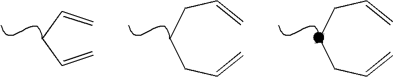
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

69. A process according to claim 68, wherein the reactive mesogen has a thermodynamically stable liquid crystal phase at room temperature.

70. A process according to claim 68, further comprising applying the reactive mesogen to the surface by a spin-coating or other solvent casting process.

71. A process according to claim 68, further comprising applying a copolymer incorporating both linear rod-like hole-transporting and photoreactive side chains to the surface.

72. A process according to claim 68, wherein the surface is a photoalignment layer.

73. A process according to claim 68, wherein the light emitting polymer is a liquid crystal uniaxially aligned by the underlying photoalignment layer surface.

74. A process according to claim 68, wherein the light emitting polymer is a liquid crystal uniaxially aligned by the liquid crystalline structure of an underlying polymer layer.

75. The process according to claim 74, wherein the underlying polymer is a charge carrier transport layer.

76. A process for applying a charge carrier transporting polymer to a surface comprising
applying a reactive mesogen to a surface: and
photopolymerizing said reactive mesogen in situ to form the light emitting polymer,
wherein the reactive mesogen has the formula: 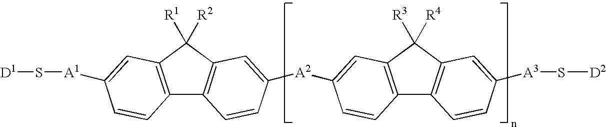
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 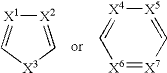
wherein one or more of X¹ and x² are independently selected from N, P, and As, and
wherein X³ may be selected from O, NH, S, PH, Se, AsH, Te, SbH, and
wherein one or more of X⁴ to X⁷ are independently selected from N, P, and As,
wherein the heterocyclic biradicals may consist of the individual rings pictured above or fused ring systems containing those heterocyclic rings,
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 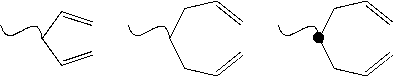
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

77. A process according to claim 76, comprising applying the reactive mesogen to the surface by a spin-coating or other solvent casting process.

78. A process according to claim 76, further comprising applying a copolymer incorporating both linear rod-like hole-transporting and photoreactive side chains to the surface.

79. A process according to claim 76, wherein the surface is a photoalignment layer.

80. A process according to claim 76, wherein the charge carrier transporting polymer is a liquid crystal uniaxially aligned by the underlying photoalignment layer surface.

81. A process according to claim 76, wherein the charge carrier transporting polymer is in the form of a liquid crystal uniaxially aligned by the liquid crystalline structure of an underlying polymer layer.

82. A process for applying a charge carrier transporting polymer to a surface comprising:
applying a reactive mesogen to a surface; and
photopolymerizing said reactive mesogen in situ to form the light emitting polymer,
wherein the reactive mesogen mixture comprises two more components at least one of which having the formula: 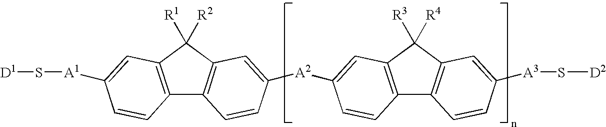
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 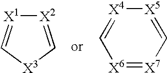
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein D¹ and D² are independently selected from the group consisting of: 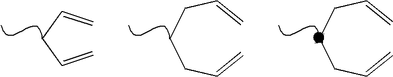
and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

83. A process according to claim 82, wherein the reactive mesogen has a thermodynamically stable liquid crystal phase at room temperature.

84. A process according to claim 82, comprising applying the reactive mesogen to the surface by a spin-coating or other solvent casting process.

85. A process according to claim 82, further comprising applying a copolymer incorporating both linear rod-like hole-transporting and photoreactive side chains to the surface.

86. A process according to claim 82, wherein the surface is a photoalignment layer.

87. A process according to claim 82, wherein the charge carrier transporting polymer is a liquid crystal uniaxially aligned by the underlying photoalignment layer surface.

88. A process according to claim 82, wherein the charge carrier transporting polymer is a liquid crystal uniaxially aligned by the liquid crystalline structure of an underlying polymer layer.

89. A compound comprising:
the following structural units: 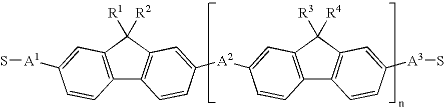
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 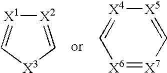
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹, R², R³, and R⁴ independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

90. A process for applying a light emitting layer to a surface comprising:
applying liquid crystalline molecules to a surface;
wherein the liquid crystalline molecules have the formula: 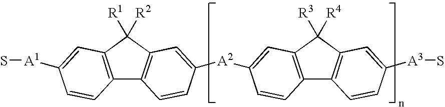
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 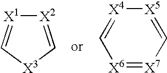
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹, R², R³, and R⁴ independently comprise branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

91. The process of claim 90 wherein the light emitting layer is a liquid crystal glass.

92. A process according to claim 90, comprising applying the liquid crystalline molecules to the surface by a spin-coating or other solvent casting process.

93. A process according to claim 90, further comprising applying a copolymer incorporating both linear rod-like hole-transporting and photoreactive side chains to the surface.

94. A process according to claim 90, wherein the surface is a photoalignment layer.

95. A process according to claim 90, wherein the light emitting layer is a liquid crystal uniaxially aligned by the underlying photoalignment layer surface.

96. A process according to claim 90, wherein the light emitting layer is a liquid crystal uniaxially aligned by the liquid crystalline structure of an underlying device layer.

97. A process for applying a charge carrier transporting layer to a surface comprising
applying liquid crystalline materials to the surface;
wherein the liquid crystalline molecules have the formula: 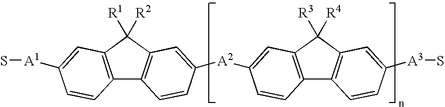
wherein A¹, and A³ are selected from a single bond, an aryl biradical, or a series of two or more aryl biradicals concatenated together in a substantially linear chain connecting the central fluorene units and flexible spacer units S, and
wherein each of n A² may independently consist of a series of one or more aryl biradicals concatenated together in a substantially linear chain connecting adjacent fluorene units or may consist of a single bond, and
wherein any one, some, or all of A¹, A², and A³ contain at least two heterocyclic aryl biradicals containing five or six-membered aromatic rings with the general formulae: 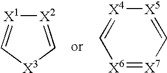
wherein one or more of X¹ and X² are independently selected from N, P, and As, and
wherein X³ may be selected from O, NH, S, PH, Se, AsH, Te, SbH, and
wherein one or more of X⁴ to X⁷ are independently selected from N, P, and As,
and wherein the heterocyclic biradicals may consist of the individual rings pictured above or fused ring systems containing those heterocyclic rings, and
wherein S are spacer groups independently comprising branched, straight chain, or cyclic alkyl groups with 3 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein R¹, R², R³, and R⁴ independently comprise branched, straight chain, or cyclic alkyl groups with 3 to 12 carbon atoms, which are unsubstituted, or mono- or poly-substituted by F, Cl, Br, 1, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms, and
wherein n=1 to 4.

98. The process of claim 97, wherein the charge carrier transporting layer is a liquid crystal glass.

99. A process according to claim 97, comprising applying the liquid crystalline material to the surface by a spin-coating or other solvent casting process.

100. A process according to claim 97, further comprising applying a copolymer incorporating both linear rod-like hole-transporting and photoreactive side chains to the surface.

101. A process according to claim 97, wherein the surface is a photoalignment layer.

102. A process according to claim 97, wherein the charge carrier transporting layer is a liquid crystal uniaxially aligned by the underlying photoalignment layer surface.

103. A process according to claim 97, wherein the charge carrier transporting layer is a liquid crystal uniaxially aligned by the liquid crystalline structure of an underlying device layer.

104. A compound comprising:
thienothiophene fused ring structural units combined with the non-conjugated diene and fluorene structural units in the following general formula:
B₁—S₁-T₁-(F-T₂)ₚ-F-T₃-S₂—B₂
wherein B₁ is a non-conjugated diene end group;
wherein B₂ is a non-conjugated diene end group;
wherein F is a fluorene functional unit having the formula: 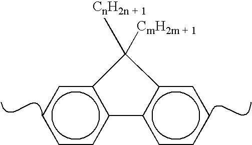
wherein n is from 1 to 10 and m is from 1 to 10;
wherein S₁ and S₂ are spacer units;
wherein at least one of T₁, T₂, and T₃ have the formula:
—W—X—Y—;
wherein X is selected from the group consisting of: 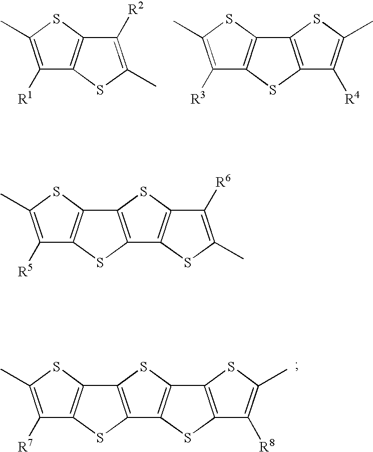
wherein W and Z are independently selected from the group consisting of: 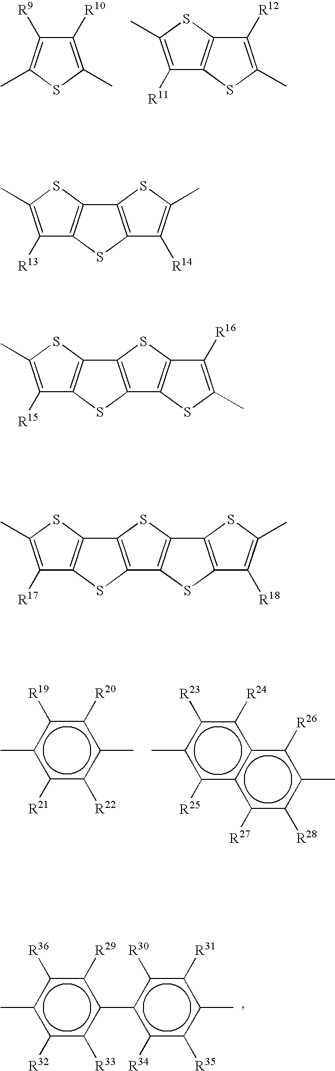
a single bond, and wherein R¹ through R³⁶ are independently selected from the group consisting of H, halogen, CN, NO₂, or branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— in such a manner that O and/or S atoms are not directly linked to each other;
wherein the T₁, T₂, and T₃ that do not have the general formula —W—X—Y— are independently selected from the group consisting of a single bond, 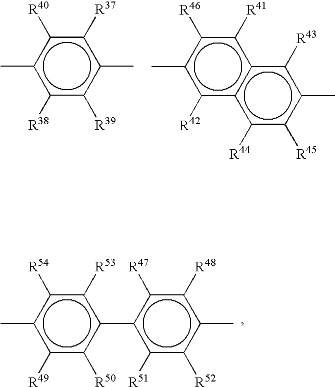
aromatic diradicals and heteroaromatic diradicals wherein R³⁷ through R⁵³ are independently selected from the group consisting of H, halogen, CN, NO₂, and branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein p=0 to 5.

105. The compound of claim 104, wherein the compound has a room-temperature nematic phase.

106. The compound of claim 104, wherein B₁ and B₂ are independently selected from the group consisting of:

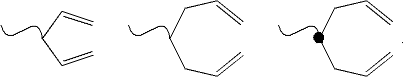

107. A process for forming a light emitting polymer comprising polymerization of a reactive mesogen having the formula:
B₁—S₁-T1—(F-T₂)ₚ-F-T₃-S₂—B₂,
wherein B₁ and B₂ are polymerizable end groups;
wherein F is a fluorene functional unit;
wherein S₁ and S₂ are spacer units; and
wherein T₁, T₂, and T₃ are thienothiophenes units.

108. The process of claim 107,
wherein at least one of T₁, T₂, and T₃ has the formula:
—W—X—Y—;
wherein X is selected from the group consisting of: 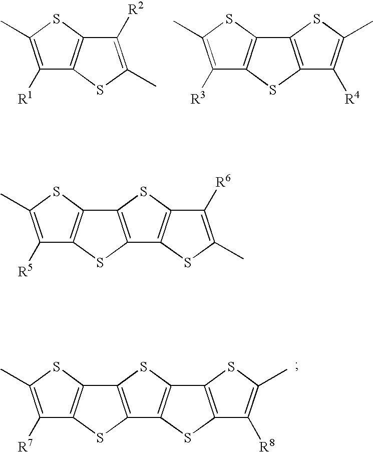
wherein W and Z are each selected from the group consisting of a single bond and 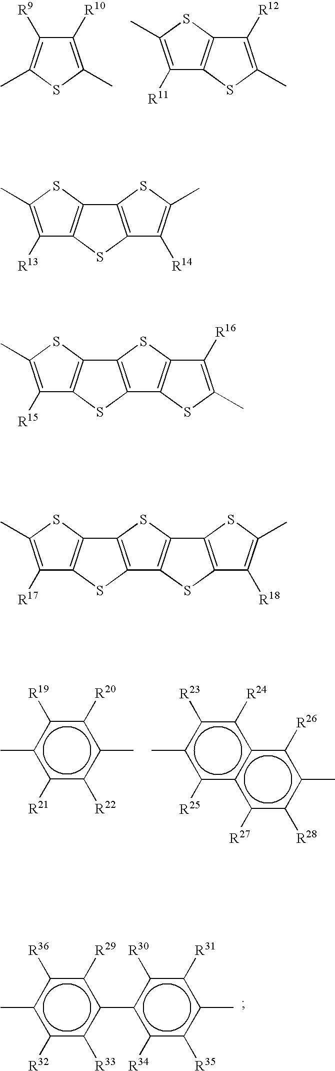
wherein R¹ through R³⁶ are independently selected from the group consisting of H, halogen, CN, NO₂, and branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms;
wherein the T₁, T₂, and T₃ that do not have the general formula —W—X—Y— are selected from the group consisting of a single bond, aromatic diradicals, heteroaromatic diradicals, and 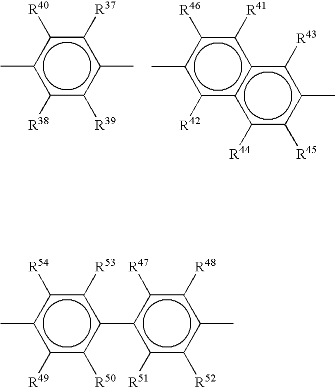
wherein R³⁷ through R⁵³ are each independently selected from a group consisting of H, halogen, CN, NO₂, and branched, straight chain, or cyclic alkyl groups with 1 to 12 carbon atoms, which are unsubstituted, or mono- or poly-substituted by F, Cl, Br, 1, or CN or wherein one or more nonadjacent CH₂ groups may be replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein p=0 to 5.

109. The process of claim 107,
wherein B₁ is a non-conjugated diene end group; and
wherein B₂ is a non-conjugated diene end group.

110. The process of claim 107,
wherein each fluorene functional unit F has the formula: 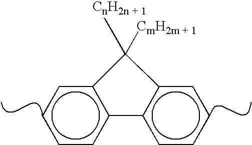
wherein n may be from 1 to 10 and wherein m may be from 1 to 10.

111. The process of claim 107, wherein B₁ and B₂ are independently selected from the group consisting of:

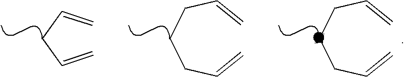

112. The process of claim 107, wherein the polymerization of a reactive mesogen is a photopolymerization.

113. The process of claim 107, wherein the photopolymerization is photoinitiator free.

114. The process of claim 107, wherein the polymer is formed from a mixture of materials.

115. A polymer comprising a reactive mesogen having the formula:
B₁—S₁-T₁-(F-T₂)ₚ—F-T₃-S₂—B₂,
wherein B₁ and B₂ are polymerizable end groups;
wherein F is a fluorene functional unit;
wherein S₁ and S₂ are spacer units; and
wherein T₁, T₂, and T₃ are thienothiophenes units.

116. The polymer of claim 115,
wherein at least one of T₁, T₂, and T₃ has the formula:
—W—X—Y—;
wherein X is selected from the group consisting of: 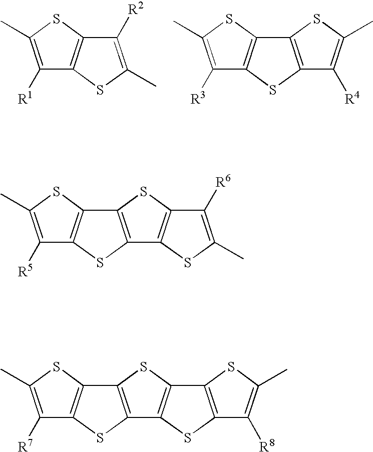
wherein W and Z are each selected from the group consisting of a single bond or: 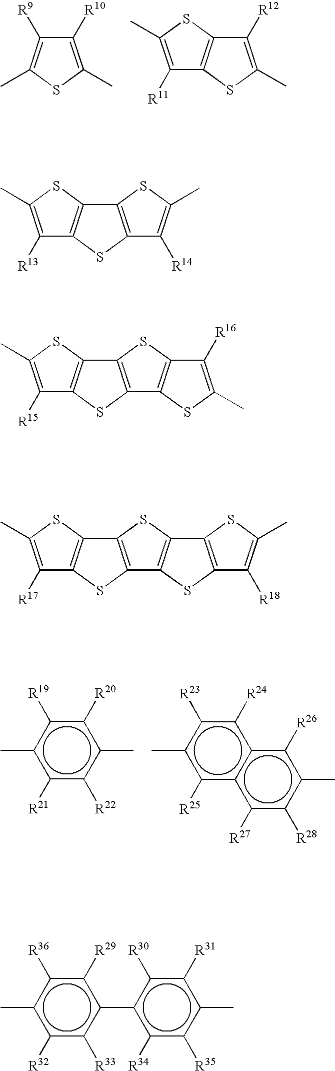
wherein R¹ through R³⁶ are independently selected from the group consisting of H, halogen, CN, NO₂, or branched, straight chain, or cyclic alkyl groups with 1 to 12 carbon atoms, which are unsubstituted, or mono- or poly-substituted by F, Cl, Br, I, or CN or wherein one or more nonadjacent CH₂ groups are replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms;
wherein the T₁, T₂, and T₃ that do not have the general formula —W—X—Y— are selected from the group consisting of a single bond, aromatic diradicals, heteroaromatic diradicals, and: 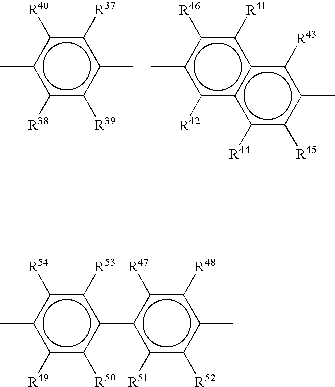
wherein R³⁷ through R⁵³ are each independently selected from a group consisting of H, halogen, CN, NO₂, and branched, straight chain, or cyclic alkyl groups with 1 to 12 carbon atoms, which are unsubstituted, or mono- or poly-substituted by F, Cl, Br, I, or CN or wherein one or more nonadjacent CH₂ groups may be replaced by —O—, —S—, —NH—, —NR—, —SiRR—, —CO—, —COO—, —OCO—, —OCO—O—, —S—CO—, —CO—S—, —CH═CH—, —C≡C— such that O and S atoms are not directly linked to other O or S atoms; and
wherein p=0 to 5.

117. The polymer of claim 115,
wherein B₁ is a non-conjugated diene end group; and
wherein B₂ is a non-conjugated diene end group.

118. The polymer of claim 115,
wherein each fluorene functional unit F has the formula: 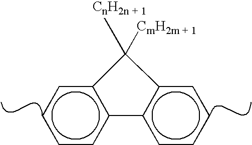
wherein n may be from 1 to 10 and wherein m may be from 1 to 10.

119. The polymer of claim 115, wherein B₁ and B₂ are independently selected from the group consisting of:

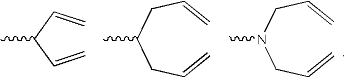

120. A device comprising a polymer layer according to claim 115.

121. The device of claim 120, wherein the device is one of an electronic device, a light emitting device, an organic light emitting device, a lighting element and a laser.

122. A polymer comprising: [chemical structure]
